(19) United States
(12) Patent Application Publication
(10) Pub. No.: US 2019/0083569 A1
(43) Pub. Date: Mar. 21, 2019

(54) MICROBICIDAL COMPOSITIONS AND METHODS FOR TREATMENT OF VIRAL INFECTIONS

(71) Applicant: University of Louisville Research Foundation, Inc., Louisville, KY (US)

(72) Inventors: Jill Steinbach-Rankins, Louisville, KY (US); Kenneth Palmer, Louisville, KY (US)

(21) Appl. No.: 16/043,602

(22) Filed: Jul. 24, 2018

Related U.S. Application Data
(63) Continuation of application No. 15/180,963, filed on Jun. 13, 2016, now abandoned.
(60) Provisional application No. 62/174,346, filed on Jun. 11, 2015.

(51) Int. Cl. A61K 38/16; A61K 31/675; A61K 31/522; A61K 47/69; A61K 47/59; A61K 45/06

(52) U.S. Cl. CPC A61K 38/16; A61K 31/675; A61K 31/522; A61K 9/0043; A61K 47/6953; A61K 47/593; A61K 45/06; Y02A 50/389

(57) ABSTRACT

A composition is provided comprising an electrospun fiber having a surface with a biological adhesive moiety conjugated to the surface of the electrospun fiber. The biological adhesive moiety included in the composition can be a lectin such as griffithsin. The composition can further include an effective amount of an antiviral agent encapsulated by an electrospun fiber. Nanoparticles including a microbicide conjugated to the surface of the nanoparticle can also be included in the composition. Methods of treating a viral infection are also provided and include administering to a subject an effective amount of a composition comprising an electrospun fiber having a surface and a biological adhesive moiety conjugated to the surface of the electrospun fiber.

### RELATED APPLICATIONS

This application is a continuation application of U.S. patent application Ser. No. 15/180,963, filed Jun. 13, 2016, and claiming priority from U.S. Provisional Application Ser. No. 62/174,346, filed Jun. 11, 2015, the entire disclosure of which is incorporated herein by this reference.

### GOVERNMENT INTEREST

This invention was made with government support under grant number U19 AI113182 awarded by the National Institutes of Health. The government has certain rights in the invention.

### TECHNICAL FIELD

The presently-disclosed subject matter relates to microbicidal compositions and methods for the treatment of viral infections. In particular, the presently-disclosed subject matter relates to microbicidal compositions and therapeutic methods that make use of an electrospun fiber and a biological adhesive moiety conjugated to the surface of the electrospun fiber.

### BACKGROUND

Sexually transmitted infections (STIs) affect 340 million new people per year, and exert a significant impact on global health. Herpes simplex virus type 2 (HSV-2) infects more than 500 million people worldwide and causes an estimated 23 million new infections each year, while HIV affects approximately 35 million people globally. Untreated STIs, such as HSV-2, can enhance both the acquisition and transmission of human immunodeficiency virus (HIV) and other STIs by 2- to 7-fold. HSV-2, specifically, is believed to increase the propensity and transmission of HIV and other STIs by: disrupting the vaginal epithelium and creating virus entry portals, recruiting HIV “target” inflammatory cells during reactivation, and increasing plasma HIV RNA levels during HSV-2 co-infection. Moreover, in contrast to previous notions of quiescent latency, infection with HSV results in frequent shedding (˜25% of days) and is often asymptomatic or undetectable (>80% of occurrences). Between the increasing prevalence of genital herpes, the frequency of clinical recurrences and asymptomatic shedding, and the association of HSV-2 with HIV co-infection, there is an urgent need for new strategies to simultaneously prevent HSV-2 and HIV infections in one delivery platform.

Despite the crucial need to develop prophylactic agents and therapeutics, vaccines and antiviral agents have only been moderately successful in preventing STIs, including HIV and HSV, and in curing STIs post-infection. In comparison, microbicides, or, in other words, biocidal compounds or substances whose purpose is to reduce the infectivity of microbes such as viruses, have the promising potential to prevent and treat STIs, while providing simultaneous female-controlled protection against a diversity of infections and unplanned pregnancy. In light of recent clinical trials, the development of a specific, multipurpose microbicide that prevents multiple STIs while increasing user adherence, is an urgent need. One way of increasing user adherence is to provide a delivery platform that requires less frequent dosing, by offering sustained-release of active agents within the vaginal tract (≥1 month). However, toward this goal, some of the current microbicide dosage form front-runners, including gels and intravaginal rings (IVRs), have their respective delivery hurdles. Gels are challenged with leakage from the vaginal cavity and remaining present to deliver active agents within the vaginal environment. Additionally, while IVRs offer longer delivery durations and durability, IVRs have only recently demonstrated co-administration of both hydrophobic and hydrophilic agents from one device. Moreover, the high temperature processing conditions often associated with IVR fabrication may prove challenging for the incorporation of biological agents.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments of the presently-disclosed subject matter, a composition is provided that comprises an electrospun fiber having a surface, and a biological adhesive moiety conjugated to the surface of the electrospun fiber. In some embodiments, the electrospun fiber scaffold is comprised of a biodegradable polymer, such as, in certain embodiments, poly(lactic-co-glycolic acid) (PLGA), poly(L-lactide-ε-caprolactone) (PLCL), polybutyl acrylate, polyacrylic acid, and combinations thereof. In some embodiments, the electrospun fiber is comprised of PLGA.

With respect to the biological adhesive moiety conjugated to the electrospun fiber, numerous molecules capable of adherence to a virus or portion thereof can be utilized. For example, in some embodiments, the biological adhesive moiety is a lectin. In some embodiments that make use of a lectin as a biological adhesive moiety, the lectin is griffithsin (GRFT) and, in some embodiments, the griffithsin is included in the composition at a concentration of about 0.00005 nmol to about 5 nmol per mg of the electrospun fiber.

To conjugate the biological adhesive moiety to the electrospun fiber, in some embodiments, the biological adhesive moiety is conjugated to the surface of the electrospun fiber with a chemical crosslinker. In some embodiments, the chemical crosslinker comprises carbodiimide.

In further embodiments of the compositions of the presently-disclosed subject matter, the compositions further comprise one or more antiviral agents including, in certain embodiments, acyclovir (ACV), tenofovir (TFV), tenofovir disoproxil fumarate (TFD), and/or combinations thereof. In some embodiments, the antiviral agent is encapsulated by the electrospun fiber. In some embodiments, the electrospun fibers included in an exemplary composition are comprised of a first electrospun fiber and a second electrospun fiber, with the biological adhesive moiety being conjugated to the first electrospun fiber and with the one or more antiviral agents being encapsulated by the second electrospun fiber. In some embodiments, in addition to or instead of the electrospun fibers, a composition of the presently-disclosed subject matter can also further comprise one or more polymer nanoparticles, with each of the one or more polymer nanoparticles conjugated to and/or encapsulating a biological adhesive moiety, an anti-viral agent, or both.

In yet further embodiments of the presently-disclosed subject matter are methods for treating a viral infection. In some embodiments, a method for treating a viral infection is provided that comprises administering to a subject an effective amount of a composition of the presently-disclosed subject matter. In some embodiments, the composition is administered intravaginally or intranasally to thereby treat the viral infection. In some embodiments, the viral infection is selected from a herpes simplex virus 2 infection, a human immunodeficiency virus infection, a hepatitis C virus infection, a middle east respiratory virus syndrome coronavirus infection, a severe acute respiratory syndrome coronavirus infection, an ebola virus infection, a human papilloma virus infection, an influenza virus infection, an enterovirus infection, a measles virus infection, a simian immunodeficiency virus infection, a human T-lymphotrophic virus infection, and a Japanese encephalitis virus infection.

Further features and advantages of the present invention will become evident to those of ordinary skill in the art after a study of the description, figures, and non-limiting examples in this document.

### BRIEF DESCRIPTION OF THE DRAWINGS

FIG. 1 is a schematic diagram showing griffithsin (GRFT)-modified electrospun fibers (EFs) adhering to human immunodeficiency virus (HIV) in the vaginal lumen or mucosa, and preventing their entry through the underlying epithelium, with the inset image showing a scanning electron microscope (SEM) image of unmodified poly(lactic-co-glycolic acid) (PLGA) EFs (scale bar represents 1 μm);

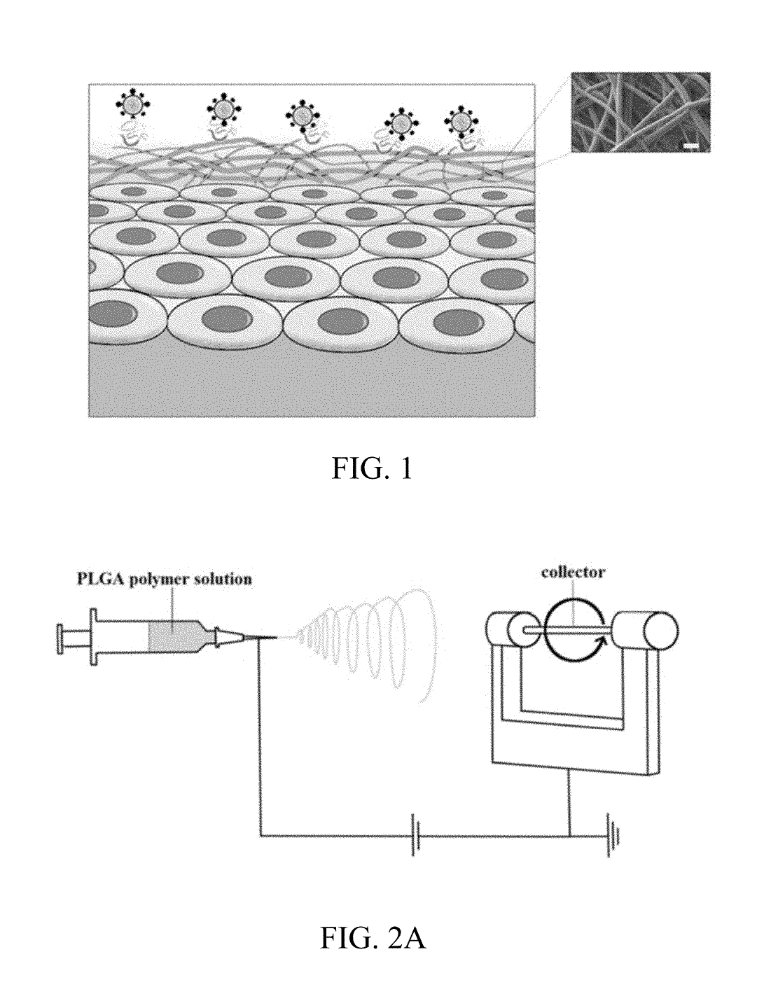

FIGS. 2A-2C include schematic diagrams showing PLGA fiber electrospinning and surface modification processes, including schematic diagrams showing: (FIG. 2A) high voltage applied to a syringe needle to thereby charge the polymer solution and cause it to erupt into a jet; (FIG. 2B) the resulting PLGA EF surface-modified with GRFT using EDC-NHS chemistry; and (FIG. 2C) GRFT-modified PLGA EF with surrounding virus;

FIGS. 3A-3E include SEM images of PLGA fibers surface-modified with (FIG. 3A) 5 nmol, (FIG. 3B) 0.5 nmol, (FIG. 3C) 0.05 nmol of GRFT per mg of fiber, and of (FIG. 3D) unmodified PLGA EFs (scale bar represents 1 μm), and further include a graph (FIG. 3E) showing diameters of unmodified (blank) and GRFT-EFs measured via ImageJ software, where no statistically significant differences in diameter were observed between formulations;

FIGS. 4A-4B include graphs showing: (FIG. 4A) the quantity of GRFT conjugated to each mg of EF fiber via (1-Ethyl-3-(3-dimethylaminopropyl)-carbodiimide)-N-hydroxysuccinimide (EDC-NHS) using 5, 0.5, 0.05, 0.005, 0.0005, and 0.00005 nmol of GRFT per mg of EF fiber; and (FIG. 4B) the quantity of GRFT released per mg of EF after 1, 2, and 4 hr incubation in simulated vaginal fluid (SVF), where negligible GRFT release was seen after 4 hr, and where both surface loading and release were measured via ELISA.

FIG. 5 is a graph showing in vitro antiviral activity of unmodified and GRFT-modified EFs in Tzm-bl cells measured via luciferase activity, where antiviral activity increases in EFs with higher surface densities of GRFT, where untreated, HIV-infected cells represent 100% infectivity, where statistical significance of each treatment group compared to untreated (no EF) is indicated by “*”, where statistical significance relative to blank EFs is indicated by “†”, and where between GRFT-EF treatment groups, all formulations demonstrated statistical significance relative to each other except the 5 and 0.5 nmol;

FIG. 6 is a graph showing in vitro antiviral activity of GRFT-EFs in Tzm-bl cells with decreasing concentrations of HIV, measured via luciferase activity, where statistical significance of each treatment group was compared to untreated (no EF) at virus dilution 1 (“*”), 2 (“†”), 3 (“‡”), and 4 (“ ”), where the overhead brackets indicate statistical significance between the antiviral activities of the GRFT EF formulations for each of the top four virus concentrations, and where the brackets denote statistical significance between individual treatment groups for virus dilution 1 (black), 2 (dark gray), 3 (medium gray), 4 (light gray);

FIGS. 7A-7C include graphs showing (FIG. 7A) VK2/E6E7, (FIG. 7B) Ect1/E6E7, and (FIG. 7C) End1/E6E7 cell viabilities measured with MTT assay after 1, 2 and 3 days EF administration, where unmodified EFs and DMSO-treated cells were the positive and negative controls for viability, respectively, and where 2 mg/mL fiber concentration was used for all treatment groups;

FIG. 8 is a graph showing in vitro antiviral activity of free GRFT in Tzm-bl cells after different durations of incubation, measured via luciferase activity;

FIG. 9 is a graph showing physical inhibition of pseudovirus penetration, where pseudovirus was administered to the apical side of a transwell membrane holding no fiber or unmodified PLGA fiber, in contact with underlying media that contained TZM-bl cells for up to 3 days, and where no HIV infection was observed for fiber samples for up to 3 days post-incubation;

FIG. 10 includes SEM images of 15 and 12% PLGA (top) and poly(L-lactide-co-caprolactone) (PLCL) (bottom) EFs, and corresponding macro pictures, where the bar represents 20 μm, where the dimensions of overall macro-fibers (right) were 0.4 cm×5 cm;

FIG. 11 is a graph showing the cumulative release of acyclovir (ACV) and tenofovir (TDF) from PLGA and PLCL EFs over one month;

FIG. 12 is an image showing that ACV PLGA and PLCL EFs prevent HSV-2 infection in Vero cells in vitro, where an HSV-2 plaque assay was performed after incubation with 1000 pfu/well HSV-2 and either (panel A) PLGA or (panel B) PLCL EFs, where the top wells were incubated with blank PLGA or PLCL fibers, where the bottom rows of each plate contain PLGA and PLCL fibers that encapsulate 1% w/w ACV, and where both PLGA and PLCL ACV EFs completely inhibited HSV-2 infection;

FIG. 13 is a graph showing dose response curves for ACV PLGA and PLCL EFs used to prevent HSV-2 infection in Vero cells in vitro, where an HSV-2 plaque assay was performed after incubation with 1000 pfu/well HSV-2 and either ACV PLGA or PLCL EFs or free ACV;

FIG. 14 is a graph showing that 20% TDF EFs prevented HIV-1 infection in TZM-bl cells in vitro, where 20% PLCL-TDF EFs were diluted to provide an assessment of dose response, and where PLCL 10% EFs are shown as an example, but all PLGA/PLCL 1, 10, and 20% EFs inhibited HIV;

FIG. 15 includes graphs showing that GRFT-EFs completely inhibit HIV-1 infection in vitro, including a graph (left) showing the density of GRFT surface-modification and a graph (right) showing the dose-dependence observed in vitro as a function of this modification, and where the top 2 concentrations are completely efficacious against HIV-1 in vitro;

FIG. 16 includes images showing that blank/bare PLGA and PLCL EFs inhibit HSV-2 penetration to underlying cells, including images showing, from left: no HSV+no fiber; HSV+no fiber; HSV+bare PLGA, and HSV+bare PLCL;

FIG. 17 is a graph showing cytotoxicity in VK2 cells, with similar cell viabilities (>93%) being seen for Ect and End cell lines but not depicted in the graph for purposes of brevity;

FIG. 18 is a graph showing that GRFT API blocks HIV-1 infection in ectocervical explants, where polarized tissue explants were set up in duplicate and exposed to GRFT as the active pharmaceutical ingredient (API) in addition to 10⁴ TC ID50 of HIV-1BaL overnight, where, after washing, the tissues were cultured for 21 days with the basolateral medium replaced every 3 to 4 days, and where the data show that the GRFT API blocked HIV-1 infection down to 10 μM in ectocervical tissue;

FIG. 19 is a graph showing the efficacy of EFs and nanoparticles (NPs) based on extract dilution (% HIV-1 Infection vs. Dilution in vitro);

FIG. 20 is a graph showing the efficacy of EFs and NPs based on extract concentration (% HIV-1 Infection vs. Concentration in vitro);

FIG. 21 is a graph showing an IC50 curve based on loading of GRFT in EFs and NPS, and showing that GRFT maintains activity in both EFs and NPs;

FIGS. 22A-22E are graphs showing tunable release of GRFT from different ratios of PLGA:PBA-co-PAA EFs with a pH change from 5 to 7.4 at 24 hrs;

FIGS. 23A-23D are graphs showing pH-tunable release of GRFT from EFs with different formulations;

FIGS. 24A-24D are graphs showing tunable sustained release of GRFT from PLGA NPs in PBS and SVF; and

FIG. 25 is a graph showing tunable sustained release of GRFT from mPEG(5000)-PLGA versus PLGA NPs in PBS.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes microbicidal compositions and methods for the treatment of viral infections. In particular, the presently-disclosed subject matter includes microbicidal compositions and methods that make use of an electrospun fiber and a biological adhesive moiety conjugated to the surface of the electrospun fiber.

The female reproductive tract is covered by mucus, a glycoprotein gel; comprised of 95% water, 1-2% mucin fibers, and trace constituents including salts, DNA, lactic acid, enzymes, and other proteins. To establish infection, a virus must travel through the mucous barrier to reach epithelial and/or sub-epithelial cells. With pore sizes larger than virus (HSV for example is approximately 170 nm, while HIV is approximately 100 nm), cervicovaginal mucus has been examined for its ability to hinder virus transport. In fact, cervicovaginal mucus is believed to provide innate protection against infection primarily through mucoadhesive mucin-glycoprotein interactions, by physically decreasing the flux of pathogens to the underlying epithelium. Similarly, cell surface heparan sulfate proteoglycan, has adhesive properties to viral glycoproteins, and is known to provide an initial contact point for herpes simplex virions to adhere to and “surf” down cell filopodia—resulting in subsequent virus fusion or endocytosis. In this regard, the presently-disclosed subject matter is based, at least in part, on the discovery of a unique delivery vehicle or scaffold that is comprised of polymeric electrospun fibers (EFs) and that includes specific adhesive and/or antiviral properties to provide virus entry inhibition.

In some embodiments of the presently-disclosed subject matter, a microbicidal composition is thus provided that includes an electrospun fiber having a surface and a biological adhesive moiety conjugated to and modifying the surface of the electrospun fiber. In some embodiments, the electrospun fibers are arranged in a scaffold. In some embodiments, the biological adhesive moiety in the composition is configured to or is capable of immobilizing and inactivating a virus.

The terms “electrospun fiber” is used herein to refer to fibers formed through the use of electric force to draw charged threads of polymer solutions or polymer melts, while the term “electrospun fiber scaffold” is used herein to refer to the arrangement of such electrospun fibers into a supporting framework that can then be used to support cells, therapeutic agents, including both biological and synthetic therapeutic agents, or other additional materials. Of course, various electrospinning methods known to those of ordinary skill in the art can be used to produce such fibers and scaffolds. For example, in some embodiments, the electrospinning techniques used to generate the fibers can make use of a variety of materials, including polymers, composites, and ceramics, but will generally make use of a high-voltage power supply, a spinneret (e.g., a hypodermic needle), and an electrically-conductive collector (e.g., a steel mandrel). To perform the electrospinning process using these materials, an electrospinning liquid (i.e., a melt or solution of the desired materials that will be used to form the fibers) is generally first loaded into a syringe and is then fed at a specific rate through a syringe. In some cases, a well-controlled environment (e.g., humidity, temperature, and atmosphere) can be used to achieve a smooth, reproducible operation of electrospinning.

As the liquid is fed through the syringe, at a sufficiently high voltage, the repulsion between the charges immobilized on the surface of the resulting liquid droplet overcomes the confinement of surface tension and then induces the ejection of a liquid jet from the orifice. The charged jet then goes through a whipping and stretching process, and subsequently results in the formation of uniform fibers. Further, as the jet is stretched and the solvent is evaporated, the diameters of the fibers can then be continuously reduced to a scale as small as tens of nanometers and, under the influence of electrical field, the fibers can subsequently be forced to travel towards the grounded collector, onto which they are typically deposited as a non-woven structure or scaffold. In this regard, by manipulating the electrical field or using mechanical force, different assemblies or scaffolds of fibers can be created. Moreover, in some embodiments, the fibers themselves can include various secondary structures, including, but not limited to, core-sheath structures, hollow structures, porous structures, and the like.

In some embodiments of the presently-disclosed subject matter, the fibers that are electrospun are comprised of a non-biodegradable polymer or, in other embodiments, the fibers that are electrospun are comprised of a biodegradable polymer. The term “biodegradable” as used herein is intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells or otherwise within the body of a subject. Degradation may occur by hydrolysis, oxidation, enzymatic processes, phagocytosis, or other processes.

Such biodegradable polymers are known to those of ordinary skill in the art and include, but are not limited to, synthetic polymers, natural polymers, blends of synthetic and natural polymers, inorganic materials, and the like. Regardless of the particular type of biodegradable polymer utilized, however, by making use of a biodegradable polymer to form an electropsun fiber and then conjugating that fiber to a biological adhesive moiety, the release of the adhesive moiety or other molecules from the electrospun fibers can be tuned, for example, to release the bound molecules after a certain period of time, at a constant, steady-state level over extended time periods, and/or at specific pH values. In some embodiments, blends of polymers are utilized to form the fibers to improve or modulate their biocompatibility as well as their mechanical, physical, and chemical properties.

In some embodiments, the fibers are comprised of: poly(lactic-co-glycolic acid) (PLGA); polybutyl acrylate; polyacrylic acid; poly(α-esters): polylactide (PLA); polyglycolic acid (PGA); poly(L-lactic acid) (PLLA); poly(D-lactic acid) (PDLA); polyurethane; polyethylene glycol; polyethylene oxide; polyvinyl alcohol; poly(methyl methacrylate) (PMMA); polystyrene (PS); polycaprolactone (PCL), including poly(L-lactide-ε-caprolactone) (PLCL); poly(propylene fumarate) (PPF); and combinations and/or blends thereof. In some embodiments, the electrospun fiber scaffold is comprised of about 15% w/w PLGA and about 12% w/w PLCL. In some embodiments, the electrospun fiber is comprised of PLGA.

In some embodiments, the polymer used to construct the electrospun fibers in accordance with the presently-disclosed subject matter can be selected from one of many polymer classes, including, but not limited to: polyanydrides; polyacetals; poly(ortho esters), polycarbonates; polyurethanes; polyphosphazenes; polyphosphoesters; polyethers (e.g., PEG, poly(propylene glycol), poly(tetrahydrofuran), and/or PEG diacrylate); proteins, such as collagen, fibrin, elastin, and/or albumin; polysaccharides, such as chitosan, chondroitin sulfate/hyaluronic acid, alginate, poly-1-lysine, etc.; and combinations and/or blends thereof.

Turning now to the biological adhesive moieties included in the compositions of the presently-disclosed subject matter, the phrase “biological adhesive moiety” and grammatical variations thereof is used herein to refer to biological molecules that are capable of binding to or otherwise adhering to other biological molecules and that are appropriate for conjugation to an electrospun fiber of the presently-disclosed subject matter based on the intended used of that electropsun fiber. For example, in some embodiments, the biological adhesive moiety is a heparan sulfate proteoglycan (HSPG) or, in other words, a macromolecule comprised of a core protein decorated with heparan sulfate (i.e., linear polysaccharide) chains. As another example, in some embodiments, the biological adhesive moiety is a lectin or, in other words, a carbohydrate-binding protein that is highly-specific for sugar moieties, including the sugar moieties present in the viral glycoproteins of many viral envelopes.

In some embodiments of the presently-disclosed subject matter, the biological agent conjugated to the surface of the electrospun fiber is the lectin, griffithsin (GRFT) or is a griffithsin-like or griffithsin-derived peptide (e.g., a tandemer). Griffithsin (GRFT) is a 12.77-kDa red-alga-derived lectin that binds the terminal mannose residues on the asparagine (N)-linked Man5-9GlcNAc2 structures that comprise the vast majority of N-linked glycans in the HIV type 1 (HIV-1) glycan shield. In this regard, it is appreciated that GRFT not only has broad-spectrum antiviral activity against HIV-1 and HIV-2, but also in an array of pathogenic coronaviruses, including, but not limited to a herpes simplex virus 2 infection, a human immunodeficiency virus infection, a hepatitis C virus infection, a middle east respiratory virus syndrome coronavirus infection, a severe acute respiratory syndrome coronavirus infection, an ebola virus infection, a human papilloma virus infection, an influenza virus infection, an enterovirus infection, a measles virus infection, a simian immunodeficiency virus infection, a human T-lymphotrophic virus infection, and a Japanese encephalitis virus infection. In some embodiments, to provide a composition capable of effectively treating an infection by the foregoing viruses, a composition is provided that comprises GRFT at a concentration of about 0.00005 nmol, about 0.0005 nmol, about 0.005 nmol, about 0.05 nmol, about 0.5 nmol, to about 5 nmol per mg of the electrospun fiber. For further information and guidance regarding the lectin, griffithsin, including its inhibitory effects on viruses, see, e.g., Nixon, B. et al. J Virol 87, 6257-69 (2013); Emau, P. et al. J Med Primatol 36, 244-53 (2007); Barton, C. et al. Antimicrob Agents Chemother 58, 120-7 (2014); Mori, T. et al. J Biol Chem 280, 9345-53 (2005), Moulaei, T. et al. Structure 18, 1104-15 (2010); Ziolkowska, N. E. et al. Structure 14, 1127-35 (2006); Ziolkowska, N. E. et al. Proteins 67, 661-70 (2007); Kouokam, J. C. et al. PLoS One 6, e22635 (2011); Zeitlin, L., Pauly, M. & Whaley, K. J. Proc Natl Acad Sci USA 106, 6029-30 (2009); Xue, J. et al. Antimicrob Agents Chemother 57, 3976-89 (2013); Meuleman, P. et al. Antimicrob Agents Chemother 55, 5159-67 (2011); O'Keefe, B. R. et al. J Virol 84, 2511-21 (2010); Ishag, H. Z. et al. Arch Virol 158, 349-58 (2013); Ferir, G. et al. AIDS Res Hum Retroviruses 28, 1513-23 (2012); Ferir, G., Palmer, K. E. & Schols, D. Virology 417, 253-8 (2011); Ferir, G., K., P. & Schols, D. Antivirals and Antiretrovirals 4, 103-112 (2012); O'Keefe, B. R. et al. Proc Natl Acad Sci USA 106, 6099-104 (2009); or Hamorsky, K. T. et al. Antimicrob Agents Chemother 57, 2076-86 (2013); each of which are incorporated herein by this reference.

With respect to the conjugation of the biological adhesive moieties, such as griffithsin, to the surfaces of the electrospun fibers of the presently-disclosed subject matter, in some embodiments, the biological adhesive moiety is conjugated to the surface of the electrospun fiber using a chemical crosslinker. The phrase “chemical crosslinker” is used herein the refer to reagents capable of directly or indirectly facilitating the chemical joining of two or more molecules by a covalent bond. Numerous types of chemical crosslinkers are known to those skilled in the art and can be utilized in accordance with the presently-disclosed subject matter, but generally make use of portions of molecules that are reactive to a specific functional group (e.g., amines, sulfhydrals, etc.) present on a target molecules (e.g., protein, DNA, etc.). For example, in some embodiments of the presently-disclosed subject matter, the chemical crosslinker comprises a carbodiimide chemical crosslinker that joins together a carboxyl group on a first molecule to an amine group on a second molecule. Accordingly, in embodiments that make use of a carbodiimide crosslinker, numerous molecules including amine groups, including proteins, nucleic acids, and the like, can effectively be conjugated to an electrospun fiber in accordance with the presently-disclosed subject matter.

In some embodiments of the presently-disclosed subject matter, in addition to or as an alternative to including a biological adhesive moiety conjugated to an electrospun fiber, the compositions described herein include one or more antiviral agents. In some embodiments, the antiviral agents that are useful in this regard include, but are not limited to, protease inhibitors, integrase inhibitors, nucleoside reverse transcriptase inhibitors, non-nucleoside reverse transcriptase inhibitors, and nucleotide/nucleoside analogs, with specific examples of such antiviral agents including cyanovirin-N, actinohivin, zidovudine, tenofovir, acyclovir, gangcyclovir, vidarabine, idoxuridine, trifluridine, levovirin, viramidine and ribavirin, as well as foscarnet, amantadine, rimantadine, saquinavir, indinavir, amprenavir, lopinavir, ritonavir, the alpha-interferon, beta-interferon, adefovir, clevadine, entecavir, and pleconaril. In some embodiments, the antiviral agent is selected from acyclovir (ACV), tenofovir (TFV), tenofovir disoproxil fumarate (TFD), and combinations thereof. In some embodiments, the antiviral agent comprises about 1% to about 20% w/w of the composition.

The antiviral agents included in a composition of the presently-disclosed subject matter can, in some embodiments, be conjugated to the surface of an electrospun fiber similar to the biological adhesive moieties describes herein above. In other embodiments of the presently-disclosed subject matter, however, the antiviral agent is encapsulated by the electrospun fiber. As would be recognized by those skilled in the art such encapsulation can occur, for example, by combining the antiviral agent with the polymer solution during the electrospinning process. In this regard, upon subsequent production and collection of the resulting electrospun fibers, the polymers comprising the fibers surround or, in other words, encapsulate the antiviral agents. As such, in some embodiments, a composition of the presently-disclosed subject matter can be provided whereby the electrospun fibers comprising the composition comprise a first electrospun fiber to which the biological adhesive moiety is conjugated and a second electrospun fiber in which one or more antiviral agents are encapsulated. In certain embodiments, the presently-disclosed subject matter can thus be directed to an integrated and multipurpose (e.g., HSV and HIV treatment) delivery approach to “trap” and inactivate viruses, while also providing a “safety net” of sustained antiviral delivery to impact other stages of viral infection. More specifically, in some embodiments of the presently-disclosed subject matter, a multipurpose microbicidal composition can be produced whereby a first component of the composition comprised of a electrospun fiber conjugated to a biological adhesive moiety (e.g., griffithsin) acts as a “virus trap” to immobilize and inactivate a virus (e.g., HSV-2 or HIV-1) prior to cell entry, while a second component of the composition comprised of an electropsun fiber and an antiviral agent provides prolonged release of the antiviral agents for longer-term pre- and post-entry inhibition against viral infection.

In some embodiments of the presently-disclosed subject matter, a composition of the presently-disclosed subject matter (e.g., a composition comprising a first electrospun fiber conjugated to a biological adhesive moiety and a second electrospun fiber encapsulating an antiviral agent) is provided that further comprises or alternatively comprises one or more polymeric nanoparticles, with each of the polymer nanoparticles conjugated to or encapsulating a microbicide. For instance, in some embodiments, polymeric nanoparticles encapsulating an amount of a biological adhesive moiety or an antiviral agent can be produced by utilizing a double emulsion solvent evaporation technique whereby a particular polymer or polymer blend is first dissolved in an appropriate solvent, and an a biological adhesive moiety or an antiviral agent is subsequently added to that initial polymer solution. The initial polymer solution can then be sonicated and subsequently added to a second polymer solution to produce a second emulsion before hardening the produced nanoparticles via solvent evaporation. As another example, in other embodiments, polymer nanoparticles conjugated to a biological adhesive moiety or to an antiviral agent can also be produced by making use of chemical crosslinkers as described above with reference to the electrospun fibers of the presently-disclosed subject matter. Of course, numerous types of polymers, including the polymers listed above as being useful for producing electrospun fibers, can also be used to produce a nanoparticle in accordance with the presently-disclosed subject matter.

Further provided, in some embodiments of the presently-disclosed subject matter are methods for treating a viral infection. In some embodiments, a method for treating a viral infection is provided that comprises administering to a subject an effective amount of a composition of the presently-disclosed subject matter (e.g., a composition comprising an electrospun fiber with a biological adhesive moiety conjugated to and modifying the surface of the electrospun fiber).

As used herein, the terms “treatment” or “treating” relate to any treatment of an infection of a subject by a virus, including, but not limited to, prophylactic treatment and therapeutic treatment. As such, the terms treatment or treating include, but are not limited to: preventing a viral infection or the development of a viral infection; inhibiting the progression of a viral infection; arresting or preventing the development of a viral infection; reducing the severity of a viral infection; ameliorating or relieving symptoms associated with a viral infection; and causing a regression of the viral infection or one or more of the symptoms associated with the viral infection. In some embodiments, the viral infection is selected from a herpes simplex virus 2 (HSV2) infection, a human immunodeficiency virus (HIV) infection, a hepatitis C virus (HCV) infection, a middle east respiratory virus syndrome (MERS) coronavirus infection, a severe acute respiratory syndrome (SARS) coronavirus infection, an ebola virus infection, a human papilloma virus (HPV) infection, an influenza virus (e.g., H5N1 and other) infection, an enterovirus (EV) infection, a measles virus infection, a simian immunodeficiency virus (SIV) infection, a human T-lymphotrophic virus infection, and a Japanese encephalitis (JE) virus infection.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently-disclosed subject matter include, but are not limited to, systemic administration, parenteral administration (including intravascular, intramuscular, intraarterial administration), oral delivery, topical administration, buccal delivery, rectal delivery, vaginal delivery, subcutaneous administration, intraperitoneal administration, inhalation, intratracheal installation, surgical implantation, transdermal delivery, local injection, and hyper-velocity injection/bombardment. In some embodiments, administering a composition in accordance with the presently-disclosed subject matter comprises intranasally or intravaginally administering the compositions as a means to reduce the infectivity of a virus.

By virtue of the controlled and sustained delivery of therapeutic agents provided by the compositions of the presently-disclosed subject matter, as well as the ability of the compositions to trap and inactivate a virus, in some embodiments, the compositions are particularly suitable for treating chronic viral infections. For instance, in certain embodiments, a composition of the presently-disclosed subject matter can be implanted within the body of a subject and allow for sustained delivery of a therapeutic agent and sustained viral control over an extended time period. In some embodiments, through such sustained delivery of a therapeutic agent and without wishing to be bound by any particular theory or mechanism, it is believed that the administration of composition of the presently-disclosed subject matter can be used to cure an individual of a chronic viral infection, such that a particular virus is no longer detected in the body of a subject.

Furthermore, in certain embodiments of the presently-disclosed subject matter, and again by virtue of the ability of the compositions to trap and inactivate a virus, a composition of the presently-disclosed subject matter is also useful in reducing the presence of viruses in various environmental conditions. For example, in some embodiments, the compositions of the presently-disclosed subject matter can be incorporated into a device, such as a surgical mask or air filter, to trap and reduce the presence of airborne viruses. As another example, in other embodiments, the compositions of the presently-disclosed subject matter may be incorporated into a suitable substrate such as a cloth or sponge and used prophylactically to remove a virus from a particular surface. Of course, as would be recognized by those skilled in the art, numerous other devices and materials incorporating a composition described herein are also envisioned to be within the scope of the presently-disclosed subject matter.

Regardless of the route of administration or particular use of a composition of the presently-disclosed subject matter, the compositions are typically administered or otherwise used in an amount effective to achieve the desired response. As used herein, the terms “effective amount” and “therapeutically effective amount” refer to an amount of the therapeutic composition sufficient to produce a measurable biological response (e.g., a reduction in viral infectivity). Actual dosage levels of active ingredients in a therapeutic composition of the presently-disclosed subject matter can be varied so as to administer or make use of an amount of the active composition that is effective to achieve the desired response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered or used, and the dose is escalated in the absence of dose-limiting toxicity to a minimally effective amount. Determination and adjustment of an effective dose, as well as evaluation of when and how to make such adjustments, are known to those of ordinary skill in the art.

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, New York; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/Philadelphia; and Duch, et al., (1998) Toxicol. Lett. 100-101:255-263, each of which are incorporated herein by reference.

As used herein, the term “subject” includes both human and animal subjects. Thus, veterinary therapeutic uses are provided in accordance with the presently-disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Polynucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. Some of the following examples may include compilations of data that are representative of data gathered at various times during the course of development and experimentation related to the present invention.

### EXAMPLES

Materials and Methods for Examples 1-4

Materials. Carboxyl-terminated 50:50 poly(lactic-co-glycolic acid) (PLGA, 0.55-0.75 dL/g, 31-57 k MW) was purchased from Lactel Absorbable Polymers (Cupertino, Calif.). Dichloromethane (DCM), dimethyl sulfoxide (DMSO), and the MTT assay kit were obtained from Sigma Aldrich (St Louis, Mo.). 1,1,1,3,3,3-hexafluoro-2-propanol (HFIP) was obtained from Fisher Scientific (Pittsburgh, Pa.). GRFT (MW 12.7 kDa) was produced in Nicotiana benthamiana as previously reported. Fetal bovine serum (FBS) and antibiotics (penicillin/streptomycin) were purchased from VWR. Dulbecco's modified Eagle's medium (DMEM) and keratinocyte serum-free medium were purchased from Invitrogen. The EDC-NHS and enzyme-linked immunosorbent assay (ELISA) kits were purchased from ThermoFisher. Simulated vaginal fluid (SVF) was prepared as described previously. The pseudovirus was produced in 293T/17 cells as previously described, using an envelope (env)-expressing plasmid (CCR5-tropic clade A strain, Q769.h5) and an env-deficient HIV-1 backbone vector (pNL4.3ΔEnv-Luc). The Env-expressing plasmid was obtained from the NIH AIDS Reagent Program.

Synthesis of Electrospun Fibers.

PLGA EFs were prepared and electrospun in HFIP with compositions spanning (8-30% wt drug/wt polymer (w/w)) to establish a baseline blank fiber formulation with well-delineated morphology. Solutions of 10-30% PLGA w/w were prepared in HFIP and allowed to solubilize overnight on a shaker at room temperature. Three milliliters of each polymer solution were aspirated into, and spun from a 3 mL plastic syringe on a custom built device housed in an air-filtered Plexiglas chamber. Flow rates spanning (0.5-3.0 mL/hr) were optimized over a range of voltages (15-27 kV) and the resulting fiber mat was collected on a rotating 25 mm outer diameter stainless steel mandrel located 25 cm from the blunt needle tip. Sample flow rate was monitored by an infusion pump (Fisher Scientific, Pittsburgh, Pa.) and the voltage was applied using a high voltage power supply (Spellman CZE 1000R). Final electrospinning conditions applied a voltage of 27 kV, and electrospun 15% w/w PLGA in HFIP with a flow rate of 2 mL/hr. After electrospinning, fibers were removed from the mandrel and dried overnight in a desiccator cabinet.

Surface-Modification of EFs with GRFT.

PLGA EFs were surface-modified with: 5, 0.5, 0.05, 0.005, 0.0005, and 0.00005 nmol GRFT per mg EF, to yield six different surface densities of the lectin GRFT on fibers. PLGA fibers were modified using EDC-NHS chemistry per manufacturer instructions. PLGA EF fiber sheets were hole-punched into circles each weighing approximately 2 mg (surface area: 28.27 mm²) and immersed in 6 mL 2-(N-morpholino)ethanesulfonic acid buffer (MES, pH 5-6) in 15 mL conical polypropylene tubes (approximately 100 hole fiber circles/tube). Two milliliters EDC (2 mg/mL IVIES) and 2 mL NHS (3 mg/mL MES) were then added to the EF. EF samples were set on a rotator for 15 minutes to activate, and were subsequently quenched with 14 μL 2-mercaptoethanol. Fibers were removed and rinsed with 1× phosphate buffered saline (PBS) (pH 7.2-7.5) to remove unreacted substrates. Stock GRFT solutions (10 mg/ml) were then diluted in 1×PBS to yield the six desired surface-modified formulations. Each GRFT dilution was added to a separate batch of EDC-activated EFs and reacted on a rotator at room temperature overnight. The following day, 2 mL of 3.5 mg/mL hydroxylamine in PBS was added to terminate the reaction, and the fibers were rinsed twice in DI water. The resulting surface-modified EFs were dried in a desiccator and stored at 4° C. until use.

Electrospun Fiber Morphology and Size.

GRFT-EF morphologies and sizes were evaluated using scanning electron microscopy (SEM). After desiccator drying, EFs were placed on carbon tape, sputter coated with gold, and imaged using SEM (Supra 35, Zeiss, Oberkochen, Germany). All SEM images were acquired at the appropriate magnifications to enable clear visualizations of the fiber microstructure. Fiber diameters were obtained by analyzing SEM images in NIH ImageJ. To determine the average diameter of each formulation, line elements were drawn across a minimum of 50 fibers per image. All experiments were conducted with a minimum sample size of n=3. Unless otherwise noted, all figure error bars represent the standard deviation of the measurement means. Statistical significance was determined using a two-sided Student's t-test (p<0.05).

Quantification of GRFT Surface-Modification Density and Surface Release.

To determine the density of GRFT on the EF surfaces, triplicates of approximately 2 mg EFs for each theoretical formulation (5, 0.5, 0.05, 0.005, 0.0005, and 0.00005 nmol GRFT per mg EF) were weighed and dissolved in 1 mL DMSO. The EF solutions were vortexed for one minute and subsequently diluted 100-fold in Tris-EDTA (TE) buffer for quantification of GRFT via ELISA.

To determine the in vitro release of “conjugated” GRFT from the EF surface, triplicate fiber pieces of approximately 2 mg were cut and immersed in 1 mL of SVF. Samples were incubated at 37° C. and constantly shaken. At fixed time points (1, 2, 4, 8, 24, 48, 72 hr, and 1, 2, 3, and 4 weeks), 1 mL of SVF from each sample was collected and replaced with another 1 mL of fresh SVF. The quantity of GRFT released from the fiber surface in the SVF was quantified with ELISA.

GRFT concentrations for surface conjugation quantification and surface release experiments were determined using a previously established ELISA method. Briefly, Maxisorp plates (Nunc) were coated with Influenza Hemagglutinin (HA) (Kentucky Bioprocessing, KBP) at 10 μg/ml in 1×PBS and incubated overnight at 4° C. Plates were blocked with 3% (w/v) bovine serum albumin (BSA) in PBS containing 0.05% Tween 20 (PBS-T). Samples were diluted 1:2 in PBS and were incubated at room temperature for 1 hr. Serial dilutions of purified GRFT were run in parallel to generate a standard curve. The HA-bound GRFT was detected by goat anti-GRFT antiserum (1:10,000) followed by horseradish peroxidase (HRP)-conjugated rabbit anti-goat IgG (1:10,000). Plates were developed with SureBlue TMB Microwell peroxidase substrate, and reactions were stopped with 1 N H2504. Absorbance was measured at 450 nm using a BioTek Synergy HT plate reader, and correlated to the free GRFT standard. Data are shown as the means±standard deviations.

Cell Lines.

TZM-bl cells were obtained from the NIH AIDS Research and Reference Reagent Program (ARRRP). These cells are a genetically engineered HeLa cell clone that express CD4, CXCR4, and CCR5 and contain Tat-responsive reporter genes for firefly luciferase (Luc) and Escherichia coli β-galactosidase under regulatory control of an HIV-1 long terminal repeat. TZM-bl cells were maintained in GIBCO DMEM containing 10% heat-inactivated FBS, 25 nM HEPES, and 50 μg gentamicin per mL in a vented T-75 culture flask. Vaginal keratinocyte, VK2/E6E7 (VK2); ectocervical, Ect1/E6E7 (Ect1); and endocervical, End1/E6E7 (End1) cell lines, obtained from ATCC, were evaluated in cytotoxicity experiments. These cell lines were selected because they are representative of the cell types in the female reproductive tract that would be exposed to topically applied EFs. VK2, Ect1, and End1 cells were maintained in keratinocyte serum-free medium (Invitrogen) supplemented with bovine pituitary extract (50 μg/ml), epidermal growth factor (0.1 ng/ml), penicillin (100 U/ml), and streptomycin (100 μg/ml). The media was further supplemented with calcium chloride (CaCl₂) to a final concentration of 0.4 mM. When cells were trypsinized for plating and single cell count, cells were neutralized using DMEM/F12 with 10% FBS, and 1% penicillin/streptomycin.

In Vitro Cytotoxicity.

VK2, Ect1, and End1 cells were plated at a density of 600,000 cells per well in 12-well plates a day before treatment, to yield 50% confluency on the day of treatment. Twenty-four hours after seeding, 3 mg unmodified and GRFT-EFs were administered to VK2, Ect1, and End1 cells for 24, 48, and 72 hours. Unmodified EFs and the top 3 concentration GRFT-EFs (0.05, 0.5, and 5 nmol/mg modified) were placed in transwell inserts in a 12-well plate with cells to assess cytotoxicity. At the corresponding time points after treatment, cytotoxicity was measured using an MTT assay. The following day, the cells were lysed and absorbance was read at 570 nm. All experiments were conducted with a minimum sample size of n=3 for each experiment. Data are shown as the means±standard deviations.

In Vitro Antiviral Activity of GRFT-EFs.

Reporter gene Env-pseudotyped virus infectivity assays were used to measure the antiviral activity of GRFT-EFs. The anti-HIV activity of the GRFT-EFs was measured as a reduction in firefly luciferase (Luc) reporter gene expression in the presence of GRFT-EFs compared to unmodified EFs after a single round of infection in TZM-bl cells. Briefly, the optimal viral input dilution of each pseudovirus (CCR5-using clade A strain Q769.h5; taken as the dilution that yielded ≥150,000 relative light units (RLU) and greater than 10 times the average RLU of cell-only background levels, as recommended in a standardized protocol), was incubated with 2 mg of the six different GRFT-EFs in triplicate at 37° C. in 96-well black solid flat-bottom culture plates. After 1.5 hr, the solution containing any unbound virus was added to freshly trypsinized cells (10,000 cells in 100 μl of growth medium containing 10 μg/ml DEAE-dextran). Additionally, one set of eight control wells received cells plus virus (no treatment, virus-infected control); another set of eight wells included cells plus virus incubated with blank fibers (fiber control); another set of eight wells included cells plus free GRFT; and the last set of eight wells received cells only (background control). After 48 hr incubation, 100 μl of culture medium was removed from each well and 100 μl of Bright Glo reagent (Promega Corp.) was added to the cells. After 2 min incubation at room temperature, luminescence was measured using the Synergy HT luminometer. The 50% inhibitory concentration (IC₅₀) was defined as the GRFT surface modification density that caused a 50% reduction in RLU compared to virus control wells after subtraction of background RLU. Data were plotted and analyzed, using one-way analysis of variance with Bonferroni post-tests (ANOVA, p<0.05), and IC₅₀ was determined using the dose-response inhibition variable slope fit in GraphPad Prism 6.0. Experiments were conducted at least in triplicate, and the data are shown as means±standard deviation.

In Vitro Penetration Assay with Unmodified EFs.

Transwell membranes within 24-well plates were removed and replaced with unmodified PLGA EFs to assess the physical potential of EFs to prevent HIV penetration. The maximum pseudovirus titer as used in antiviral efficacy experiments above was administered to the apical side of each transwell sample (with membrane removed) that contained either no fiber or PLGA fiber. Fibers were in contact with underlying media that contained TZM-bl cells, for up to 3 days. The amount of HIV infectivity relative to no treatment (no fiber in empty transwell membrane) is shown after 3 days.

In Vitro Antiviral Activity of GRFT-EFs with Increasing Virus Dose.

Two milligrams of the 0.05, 0.5, and 5 nmol per mg GRFT-EFs were incubated with ten dilutions of pseudovirus (series dilutions, threefold stepwise) beginning at the optimal viral input dilution (described above) in triplicate for 1.5 hr at 37° C. in a total volume of 100 μl growth medium in 96-well black solid flat-bottom culture plates. The antiviral activity of GRFT-EFs was assessed using TZM-bl cells as described above. Data are shown as the means±standard deviation. Data were plotted and analyzed in GraphPad Prism 6.0, using one-way analysis of variance with Bonferroni's post-tests (ANOVA, p<0.05).

### Example 1—Electrospun Formulations Form Well-Delineated, Micron-Sized Fibers

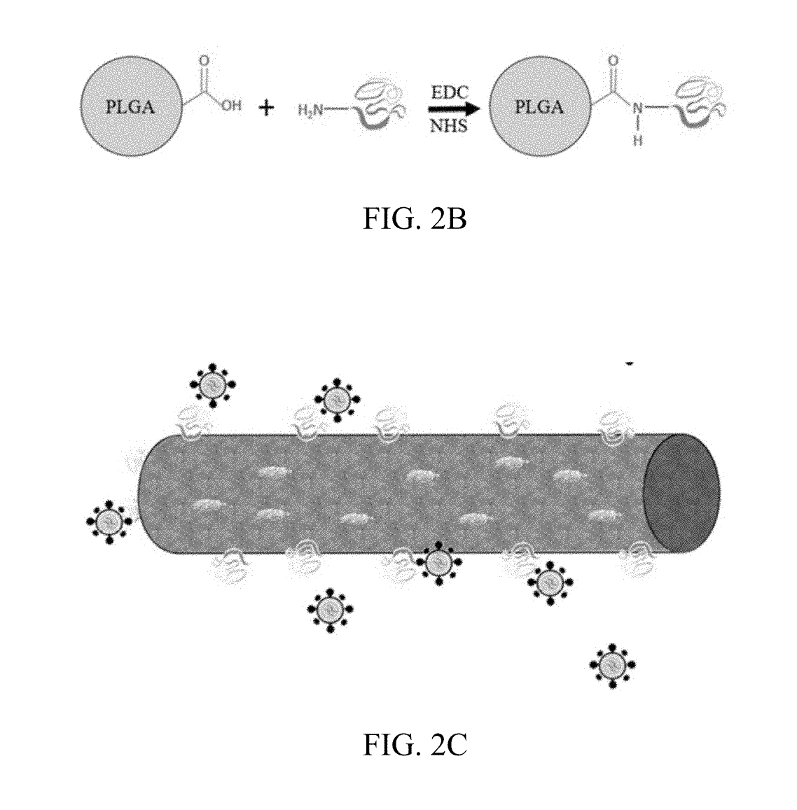

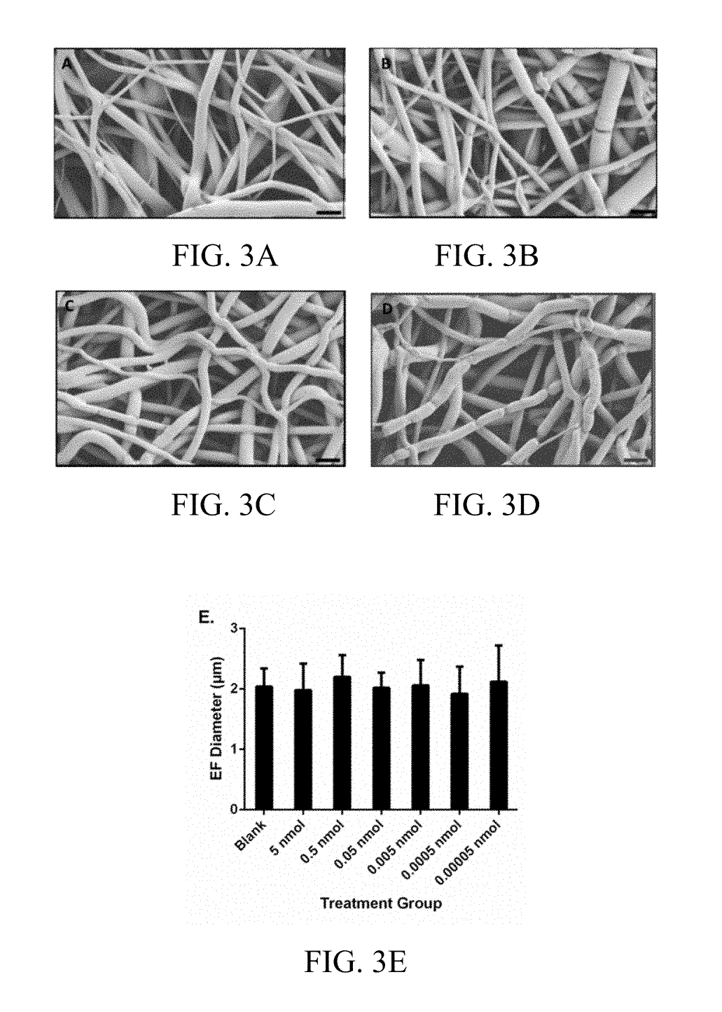

A schematic of the electrospinning process is shown in FIG. 2A. Using carbodiimide crosslinker chemistry, six different amounts of GRFT were conjugated to unmodified EFs (FIGS. 2B-2C). The morphologies of EFs with different surface modifications are shown in FIGS. 3A-3D. The average EF diameters were measured using ImageJ and are shown in FIG. 3E. Relative to unmodified EFs (FIG. 3A), the three highest modifications: 5, 0.5, and 0.05 nmol GRFT (FIGS. 3B-3D) displayed no distinct morphological differences. No statistically significant differences in diameters were found between GRFT-modified and unmodified EFs, indicating that GRFT modification does not impact EF diameter.

### Example 2—Range of GRFT Surface-Modification Densities and GRFT Release from the EF Surface

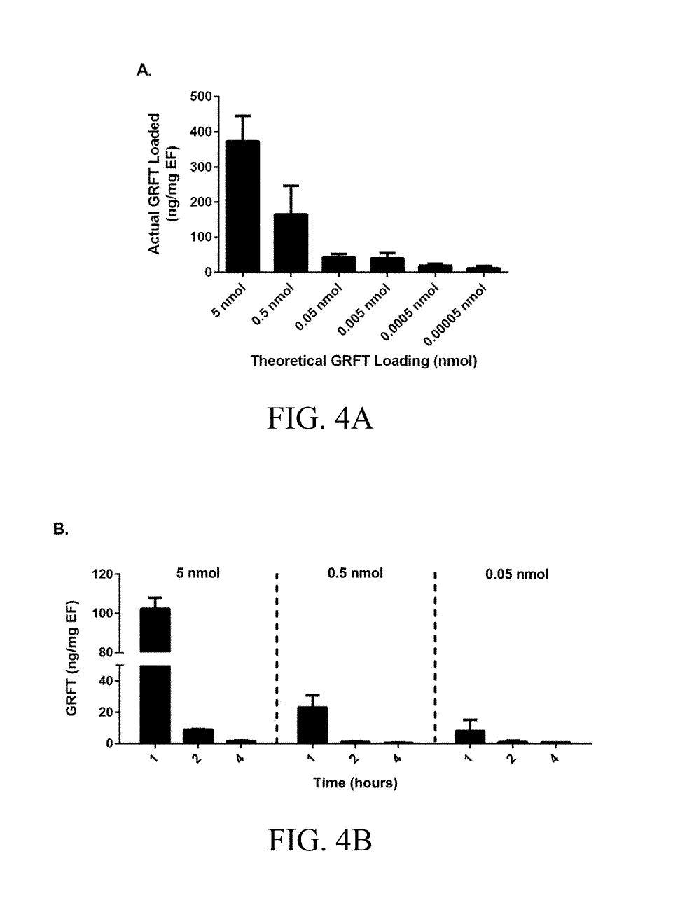

To determine the density of GRFT on the EF surface, GRFT was extracted from each of the GRFT-EFs and the amount extracted was measured using an ELISA assay (FIGS. 4A-4B). It was found that the amount of extracted GRFT positively correlated with the theoretical modification density; however, at high conjugation concentrations of GRFT, very low conjugation efficiency was achieved (Table 1). For 5, 0.5, 0.05, and 0.005 nmol/mg EF modifications, conjugation efficiencies spanning 0.6, 4.2, 6.9, and 43.2% were achieved, corresponding to 373, 165, 42, and 40 ng GRFT per mg EF. For the two lowest concentrations of modification (0.0005 and 0.00005 nmol/mg EF), the amount of GRFT was within the threshold of our detectable limit using ELISA.

TABLE 1
Amount of GRFT loaded (extracted) and released per mg of EF.
The amount of GRFT loaded increases with higher theoretical
GRFT conjugation density.

| Theoretical | | Total |
|---|---|---|
| GRFT Conjugation | Total GRFT | GRFT Released from EF |
| Density | Extracted | Surface |
| (nmol GRFT/mg EF) | (ng GRFT/mg EF) | (ng GRFT/mg EF) |
| 5 | 373 | 113 |
| 0.5 | 285 | 25 |
| 0.05 | 42 | 10 |
| 0.005 | 40 | None detected |
| 0.0005 | 19 | None detected |
| 0.00005 | 12 | None detected |

The amount of GRFT released from the GRFT-EFs was next determined as a function of time. After incubating the fibers in SVF for up to 4 hr, it was observed that 30%, 9%, and 24% of GRFT detected from loading experiments (conjugated or adsorbed to the fibers) was released into the SVF from the 5, 0.5, and 0.05 nmol/mg EFs, respectively. Negligible GRFT was detected in SVF for all formulations after 4 hr and for GRFT modification concentrations less than 0.05 nmol/mg. This demonstrated that the majority of the GRFT is conjugated to and retained on the fibers; whereas potentially adsorbed GRFT is released during the first 4 hr.

### Example 3—GRFT-EFs Demonstrate Efficacy Against HIV-1 In Vitro

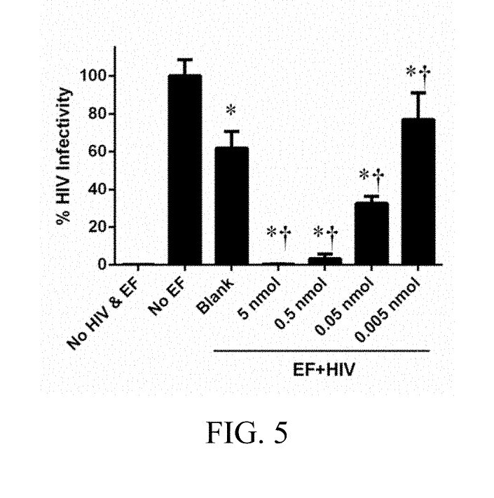

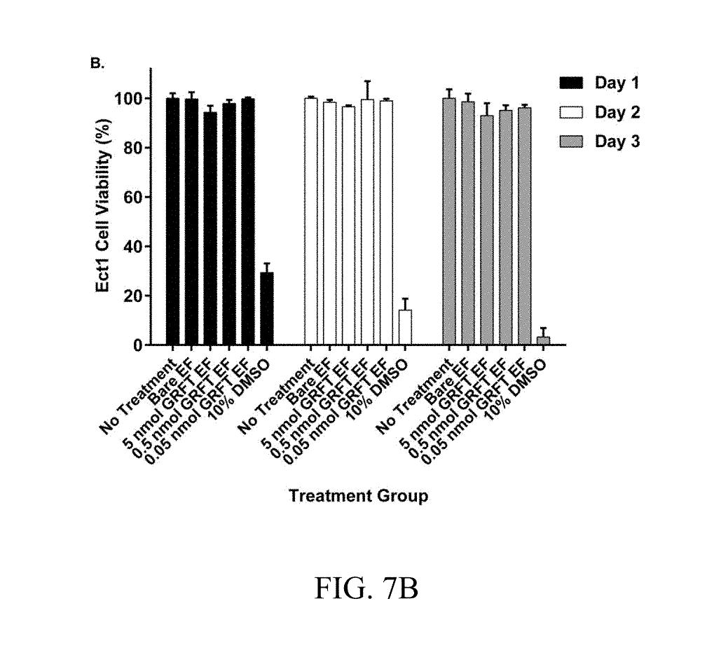

To test the antiviral activity of GRFT-EFs, the HIV-1 neutralization activity of all six surface-conjugated GRFT-EF formulations was assessed. As shown in FIG. 5, a dose-dependent effect on HIV inhibition was observed with respect to the amount of GRFT surface modification. Fibers with higher theoretical surface densities of 5 and 0.5 nmol per mg EF almost completely neutralized HIV-1 infection (100 and 97% respectively); whereas fibers with 0.05 and 0.005 nmol/mg theoretical GRFT neutralized 67 and 23% of virus infection. Unmodified EFs inhibited HIV by approximately 38%; whereas the two lowest concentrations of fibers demonstrated negligible inhibition. Using this data, the IC₅₀ of the GRFT-EFs was determined to be 41.5±2.0 ng GRFT/mg EF. Similarly the efficacy of free GRFT was evaluated as a baseline for comparison (FIG. 8), and achieved an IC₅₀ of 233±2.7 ng GRFT/mL under similar administration durations.

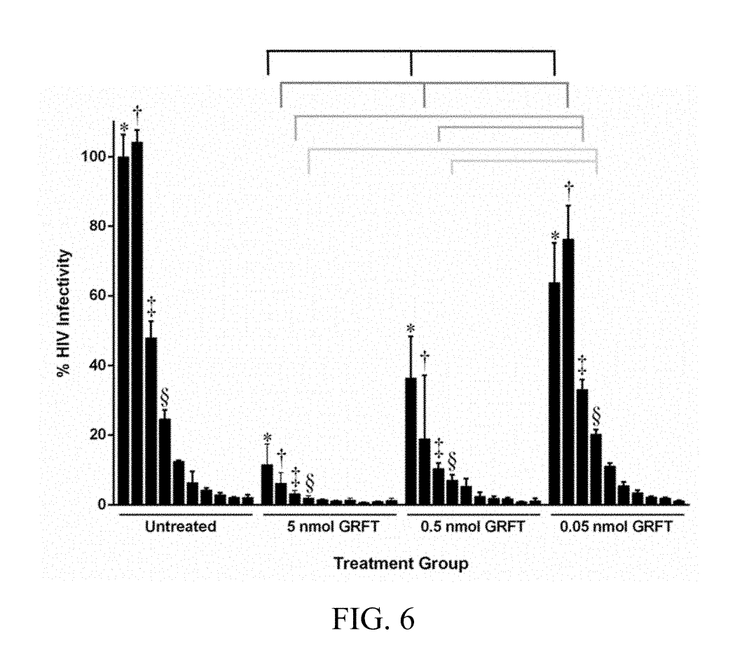

To assess the antiviral activity of these GRFT-EFs when exposed to different concentrations of HIV in vitro, their antiviral effect was evaluated across 10 different viral inoculum concentrations. As shown in FIG. 6, the top two concentrations of virus inoculum were the most viable, and provided the most insight on the ability of our EFs to inhibit infection, relative to untreated groups. EFs with a theoretical GRFT surface density of 5 nmol per mg inhibited between 89 and 99% of HIV depending on the virus concentration used. In comparison, 0.5 and 0.05 nmol inhibited between 64 to 99% and 36 to 99%, respectively. Taken together, these results indicated that GRFT-EFs were effective in inhibiting HIV-1 in a dose-dependent manner across a range of virus concentrations in vitro.

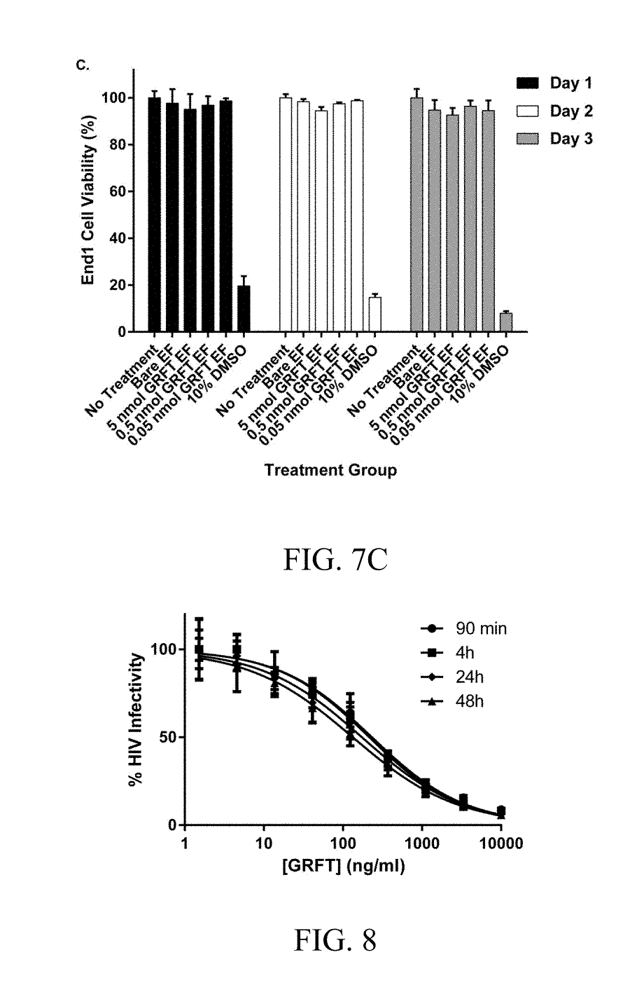

Lastly, as it was noted that blank fibers alone inhibited infection, the ability of GRFT EFs to physically prevent HIV infection was assessed. To evaluate that, transwell membranes were replaced with unmodified EFs, and virus was administered to the apical surface of PLGA EFs that had their basal sides in contact with underlying media containing TZM-bl cells. It was discovered that unmodified PLGA EFs completely inhibited HIV penetration for up to 3 days under those administration conditions in vitro (FIG. 9).

### Example 4—GRFT-EFs are Non-Toxic to Vaginal Epithelial Cells In Vitro

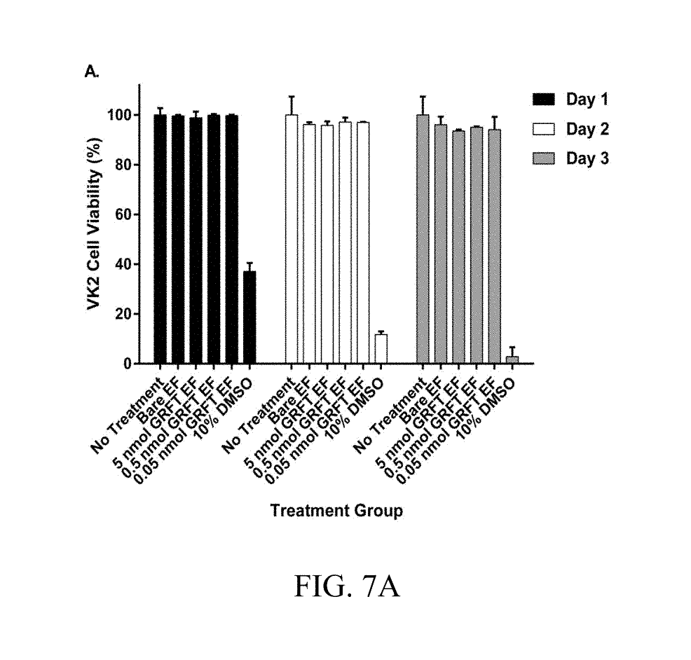

To determine the biocompatibility of GRFT-EFs and to conclude that the antiviral effects of GRFT-EFs were not attributed to fiber-induced cytotoxicity, vaginal epithelial cell lines VK2, Ect1, and End1 were used to assess the cytotoxicity of unmodified and GRFT-EFs in vitro. As shown in FIGS. 7A-7C, GRFT-EFs demonstrated greater than 94, 95, and 93% viability on days 1, 2, and 3 respectively, across all cell lines, relative to negative and positive controls of no treatment and 10% DMSO. No statistically significant changes were seen in cell viability across cell lines and duration of incubation.

Discussion of Examples 1-4

A significant obstacle to more efficacious microbicide delivery is the paucity of suitable biologic and drug delivery vehicles, targeted to the unique microenvironment of the female reproductive tract. For intravaginal delivery, cervicovaginal mucus has been studied for its ability to hinder virus transport, as it provides innate protection against infection primarily by mucoadhesive interactions, not sterics. In this regard, the natural host defense process was considered in the design of the above-described materials and it was thought that a delivery platform that incorporates similar viral-adhesive characteristics, yet with specificity for viral glycoproteins, may prevent infection by physically decreasing the flux of pathogens to the epithelium. However, just as cervicovaginal mucus poses an impediment to incoming pathogens, it can adversely affect the efficacy and transport of drug delivery. Therefore, delivery vehicle design must consider mucosal properties such that the carrier can traverse or remain stable in mucus, while ideally inhibiting virus penetration, entry, and simultaneously delivering agent to target sites (virus or host cells).

Currently, most delivery vehicles provide active agent delivery—typically ARVs—to the female reproductive tract in the form of a gel or via IVRs for long-term delivery. More recently, EFs have emerged to similarly deliver ARVs against HIV and other sexually transmitted infections. These delivery strategies highlight a traditional delivery approach of encapsulating active agents within the delivery vehicle to sustain active agent release.

However, while EFs are being developed to encapsulate active agents within the delivery vehicle, one overlooked advantage of EF scaffolds is that their surfaces may also be utilized to provide protection and delivery of active agents. Furthermore, biologic agents such as the antiviral protein, GRFT, also hold promise as alternatives to traditionally administered small molecule ARVs. With these considerations, one goal in the above-described work was to evaluate the potential of surface-modified PLGA EF scaffolds against HIV infection in vitro. Without wishing to be bound by any particular theory or mechanism, it was believed that PLGA EFs surface-modified with the potent and broad-spectrum antiviral protein, GRFT, can protect against HIV infection in vitro, by incorporating both a chemical and physical barrier to adhere to and inactivate HIV. The goals here were to create and characterize GRFT-modified EFs, evaluate how surface modification impacted HIV infection in vitro, and to assess the cytotoxicity of these EFs in vaginal epithelial cells.

The first factor that was evaluated in the design of the EFs was fiber morphology (FIGS. 3A-3E). It was observed that EF morphology remained unchanged after surface-modification with a variety of GRFT concentrations. Fiber diameters averaging approximately 2 were obtained for all groups, with no statistically significant difference observed between GRFT-EF groups.

The next thing that was characterized was the loading efficiency of GRFT on the EF surfaces. An increase in conjugated GRFT was observed with respect to theoretical GRFT concentration added. However, it was also observed that the efficiency of conjugation was rather low at high theoretical GRFT modification (5 nmol per mg EF), resulting in approximately 1% modification efficiency for 5 nmol EFs and 4-7% efficiency for 0.5 and 0.05 EFs (FIG. 4A/Table 1). In contrast, when the concentration of GRFT was decreased to 0.005 nmol per mg EF, 43% modification efficiency was obtained. At concentrations lower than 0.005 nmol per mg EF, the amount of GRFT on the fiber surface was undetectable via ELISA.

Without wishing to be bound by any particular theory, it was believed that that range of conjugation efficiencies may be attributed to a number of factors. First, the GRFT used in the study has N-terminal acetylation; thus, hindering the conjugation potential of the N-terminal amine group. Second, there are only two lysine residues, Lys6 and Lys99, in the primary sequence of GRFT (i.e. four per GRFT dimer molecule), of which the latter is buried near the interface between two monomers of the domain-swap dimer structure. Hence, there may be steric hindrance of two of the four lysine residues within the GRFT protein, resulting in insufficient primary amine groups available for conjugation. It was suggested that this may limit efficient conjugation with the fiber carboxyl groups. To overcome those challenges, one option can be to modify GRFT with an amine at a different location that is more accessible for EF conjugation. Also, it can be possible to potentially modify GRFT with amines at different locations, while also ensuring that GRFT antiviral activity is maintained. Conversely, the number or type of functional groups on PLGA EFs can be altered, or noncovalent attachment of GRFT to the EF surface can be considered to increase conjugation efficiency. Both GRFT and polymer-modification approaches can be examined to enhance the conjugation efficiency.

While one goal was to covalently modify EFs with GRFT, it was further noted that the negative charge of GRFT (pI=5.4), may induce surface adsorption of GRFT—versus covalent binding—to the hydrophobic EF surface. Therefore, to characterize the potential release of transiently surface adsorbed GRFT, a sustained release assay was performed over 4 weeks. FIG. 4B shows that the release, of what was believed to be adsorbed GRFT, from the EF surface occurs within the first 4 hr. During this time, approximately 113, 8.9, and 1.6 ng of GRFT were released from the EF surface, corresponding to 30, 9, and 24% release for the 5, 0.5, and 0.05 nmol per mg EFs. After 4 hr, negligible GRFT release was detected.

With respect to the impact of this released (or surface desorbed) GRFT on HIV infection, GRFT has been shown to immediately inactivate virus upon contact. Furthermore, in the studies HIV was pre-incubated with fiber for 90 min, and the supernatant solution (containing unbound HIV and some fiber-released GRFT) was subsequently added to cells. Despite released GRFT or unbound virus, when the solution was added to cells, full inhibition of HIV infectivity was observed in vitro for 2 EF formulations, indicating unbound GRFT (and potentially unbound virus) does not detract from efficacy.

After GRFT-EF characterization, the efficacy of our 6 surface-conjugated formulations to inhibit HIV in vitro was next assessed (FIG. 5). Similar to loading data and regardless of conjugation efficiency, a concentration-dependent increase in inhibition corresponding with GRFT surface-modification density was observed. Two milligrams of the 5 and 0.5 nmol GRFT EFs provided complete protection against HIV; whereas the same quantity of the 0.05 and 0.005 nmol per mg EFs provided 67 and 23% inhibition, respectively. Concentrations lower than 0.005 nmol per mg EF had negligible effect on HIV inhibition. From this data, an IC₅₀ of 41.5 ng GRFT per mg fiber was calculated as the amount required to inhibit HIV infection. Comparing this value to previous literature measurements of 157 ng/mL using the same virus strain (Q769.h5) (55) and with the presently-disclosed data, a more than 3-fold increase in GRFT efficacy was achieved from administration of surface-modified EFs, relative to free GRFT. This data indicated that the multivalency of fiber-bound GRFT may enhance the avidity between GRFT and viral glycoproteins, thereby resulting in more potent protection.

Another interesting observation was that blank fibers alone demonstrated a 38% decrease in HIV infection, suggesting the potential for unmodified-EFs to physically inhibit HIV infection. This is consistent with other studies that observed HIV binding to polystyrene fibers and obstruction of sperm transport through fiber meshes. Here it was suggested that HIV debilitation may be attributed to the large surface area and web-like, tortuous microstructure of fiber meshes available for HIV binding. In addition, the interaction between virus surface proteins and hydrophobic fibers, can result in an inability to detach from, or pass through fibers to infect cells.

The ability of the EFs to prevent virus penetration when virus was applied to the top of the fiber suspended in a transwell plate (FIG. 9) was also evaluated. Virus was administered to a transwell plate (with membrane removed) that contained either no fiber or PLGA fiber, in contact with underlying media that contained TZM-bl cells, for up to 3 days. It was discovered that unmodified PLGA EFs inhibited HIV penetration in vitro for up to 3 days by acting directly with virus. From that physical inhibition study, it was believed that the physical nature of multilayered porous and hydrophobic unmodified-EFs may additionally promote virus immobilization or lack of penetration, and may contribute to the ability of unmodified (and modified) EFs to inhibit HIV-1 infection in vitro, if virus is directly in contact with the EFs. From the present efficacy data, it was observed that a combination of both chemical and physical effects may play a role in garnering virus protection.

In parallel with the above work, the efficacy of EF formulations was also evaluated with various virus concentrations (FIG. 6). Similar efficacy was observed when evaluating the effect of these EFs with HIV virus dilutions. While only two virus concentrations produced 100% infection in untreated cells, the five concentrations of virus inoculum produced full or partial infections. Furthermore, for these relevant virus inocula, a surface density-dependent response in HIV infectivity was observed with our 5 nmol EF formulation completely inhibiting infection, and the 0.5 nmol group significantly inhibiting HIV infection.

Last, the modifications of GRFT-EFs were evaluated in VK2, End1, and Ect1 cell lines at days 1, 2 and 3 to assess cytotoxicity (FIG. 7). 2 mg/mL GRFT-EF of the 5, 0.5, and 0.05 nmol per mg GRFT-EFs were administered to all cell lines. It was observed that all EFs were non-toxic across the 3 vaginal cell lines, with greater than 93% viability, indicating their potential for future translation in vivo.

From this work, it was demonstrated that GRFT-EFs potently inhibit HIV infection in vitro, in addition to providing a non-toxic platform for future translation. Additionally, GRFT-EFs can provide a unique and potent way to mitigate infection, while providing the potential to combine EF surface-modification strategies with encapsulated agents in the future. A “smart” topically applied microbicide such as this, can be customized to bind to a variety of pathogen targets (e.g. HIV gp120), and release multiple agents that disrupt diverse viral infections (e.g. genetic agents, small molecule drugs, or antivirals) with the potential to provide delivery options to the microbicide and infectious disease communities.

### Example 5—Synthesis and Characterization of Electrospun Fibers (EFs) that Release Known HSV-2 and HIV-1 Antivirals: ACV and TDF

As described herein, the development of different polymer materials (initially PLGA and PLCL), while incorporating surface-modification with the biological lectin GRFT known to debilitate virus, provided a unique technology to offer sustained-release and prolonged protection against HSV-2 and HIV-1 infection. In this regard, PLGA and PLCL EFs were also successfully synthesized to efficiently encapsulate ACV and TDF, and provide sustained-release for 1 month. Fabricated EFs were made from two different polymer materials—PLGA and PLCL—that encapsulate ACV as a model antiviral against HSV-2 infection. A variety of polymer formulations were synthesized, with and without ACV encapsulation, to establish those fibers with the best processing yield and morphology. Briefly, solutions of PLGA 10-30% w/w and PLCL 8-12% w/w were prepared in various solvents (HFIP, DCM, CHCl₃, DMF:CHCl₃) and allowed to completely solubilize overnight. The next day, each polymer solution was electrospun from a plastic syringe on a custom built device housed in an air-filtered Plexiglas chamber. Flow rates were optimized over a range of voltages and the resulting fiber mat was collected on a rotating 4 or 26 mm outer diameter stainless steel mandrel. Original fibers were spun with the geometry designed for insertion into the female mouse reproductive tract (FMRT).

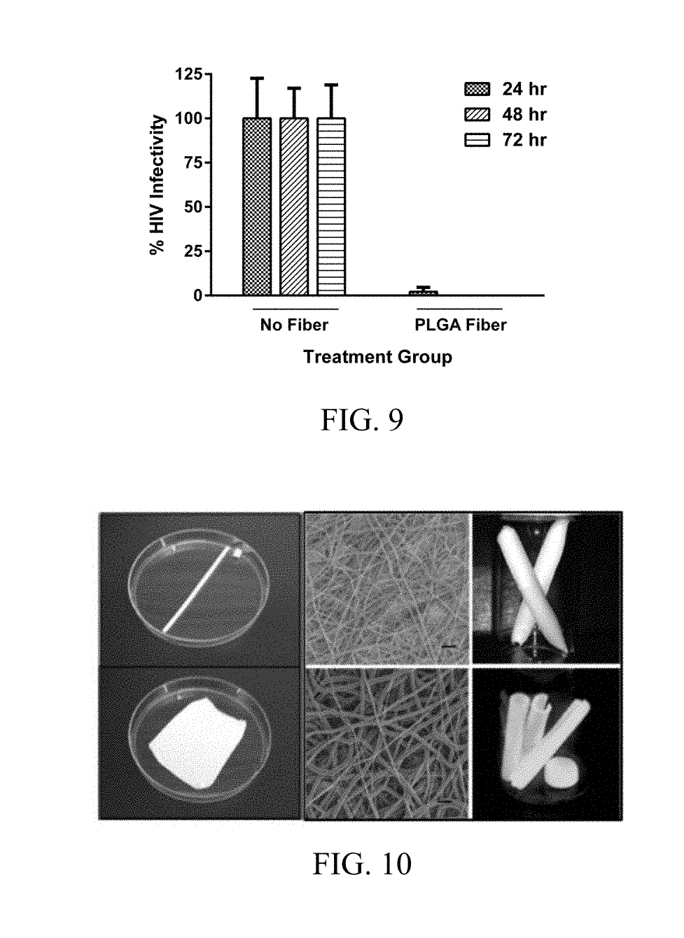

Based on scanning electron microscopy (SEM) images (see FIG. 10), it was established that 15% PLGA and 12% PLCL w/w polymer-solvent solutions in HFIP provided cohesive and well-formed fibers with diameters ranging from 0.5-2.0 μm. Using those formulations, fibers containing 1, 10, and 20% w/w ACV or TDF were electrospun, resulting in EFs containing 3-35 mg ACV and 2-70 mg TDF per fiber (˜600 mg total). The loading and controlled release of ACV and TDF from the fibers was assessed. Loading efficiencies spanning 70-80% ACV and 43-75% TDF for both 15% PLGA and 12% PLCL EFs, were achieved.

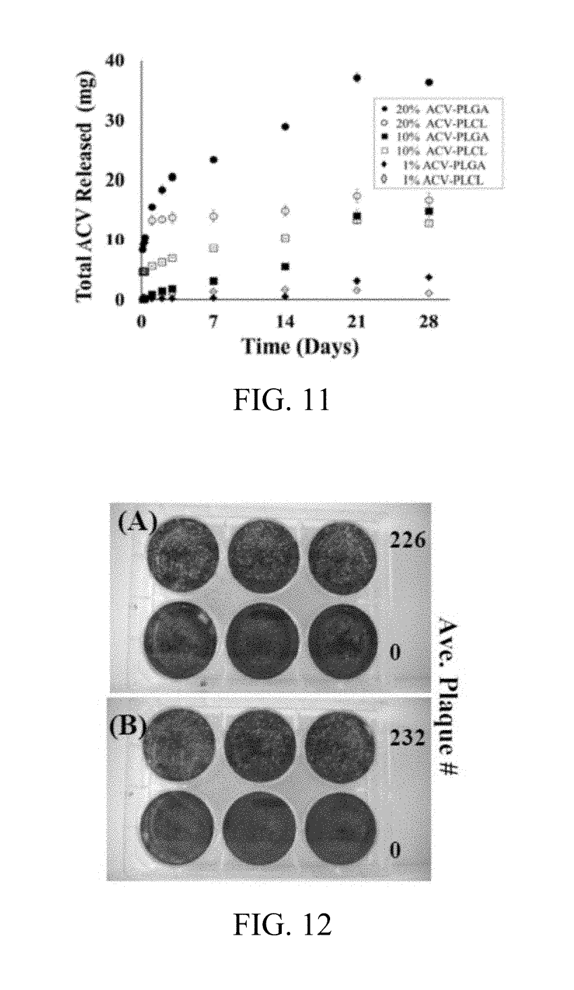

To characterize ACV and TDF release from the fibers, sections of both PLGA and PLCL fibers were incubated in simulated vaginal fluid (SVF) for one month. Eluant was taken at multiple time points, and the ACV release profiles from both the PLGA and PLCL EFs are shown in FIG. 11. Both PLGA and PLCL EFs demonstrated sustained-release of ACV and TDF after one month. In parallel, the change in EF morphology and fiber degradation over time was evaluated in SVF (images not shown due to brevity). Between 1-3 days, PLGA and PLCL EFs exhibit slight degradation spanning approximately 2.9-8.6 wt %. After 28 days the average mass loss was similar, spanning only 12-14%, with no statistically significant differences between PLGA and PLCL degradation. Both polymers maintained well-formed fiber morphology. These results indicated the potential of those EFs to provide long-term efficacious release under conditions similar to the FRT.

### Example 6—Further Evaluation of Surface Modification of EFs with the Antiviral Lectin, Griffithsin (GRFT-EFs)

In this experiment, EFs were again successfully surface-modified with GRFT. For most HIV-1 isolates, the in vitro inhibitory activity (50% effective concentration [EC₅₀]) of GRFT is in the mid-pM to low-nM range. Furthermore, efficacy after subcutaneous administration in mice has been shown, demonstrating that serum from GRFT-treated animals retained antiviral activity against HIV-1-enveloped pseudovirus in a cell-based neutralization assay. Against HSV-2, it has also been shown that GRFT has a cell-dependent EC50 ranging from 2.3-143 μM in vitro, and inhibits HSV-2 infection after a single 20 μL dose of 0.1% GRFT gel in vivo. GRFT modestly inhibited HSV-2 cell entry, while completely inhibiting cell-to-cell spread in vivo. As such, and as indicated above and without wishing to be bound by any particular theory, it was believed that modifying the surface of EFs with GRFT would similarly inhibit HIV-1 and HSV-2 infection, while additionally providing physical hindrance to virus infection and a durable antiviral sustained-delivery platform.

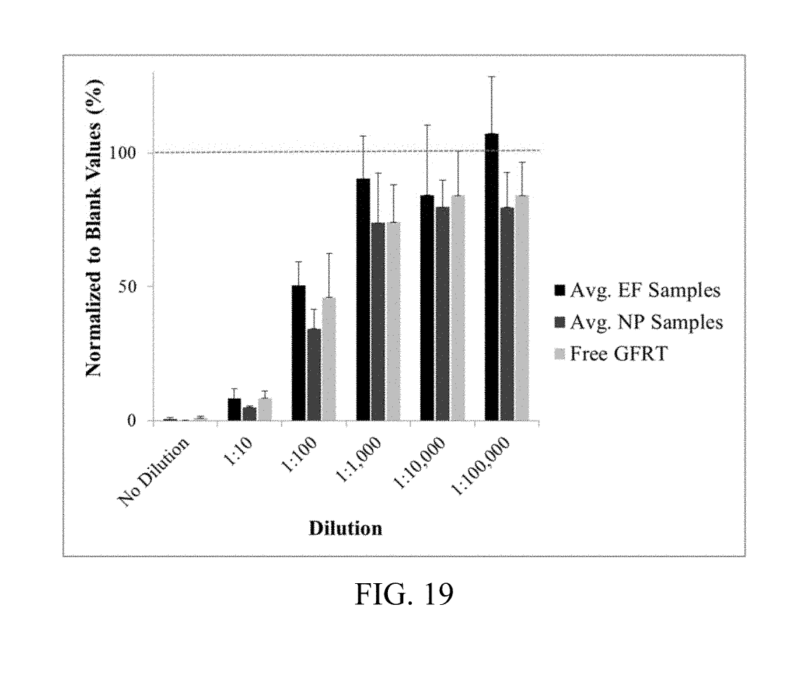

In this further experiment, EF surfaces were again modified with GRFT and the resulting surface density of GRFT was characterized. GRFT surface modification was conducted at room temperature using the EDC-NHS reaction kit to assess the lower and upper conjugation limits of the fibers. The resulting surface density was quantified using the GRFT-ELISA detection kit with a free (unconjugated) GRFT standard. The loading of GRFT on EFs was compared to its dose-dependent inhibitory effect in FIG. 15. In addition, to validate that GRFT remains conjugated to the EF surface, GRFT release experiments were conducted over one month. During the course of 28 days, only a small fraction (10%) of “loaded” GRFT was released from the EF surface—all within the first hour. This was attributed to adsorbed (vs. covalently bound) GRFT, and demonstrated the potent efficacy of these EFs, despite this minor amount of surface desorption (see FIG. 15).

### Example 7—In Vitro Cell Assays and EpiVaginal™ Tissue Models to Evaluate the Safety and Efficacy of Individual and Combined EFs Against HSV-2 and HIV-1 Infections

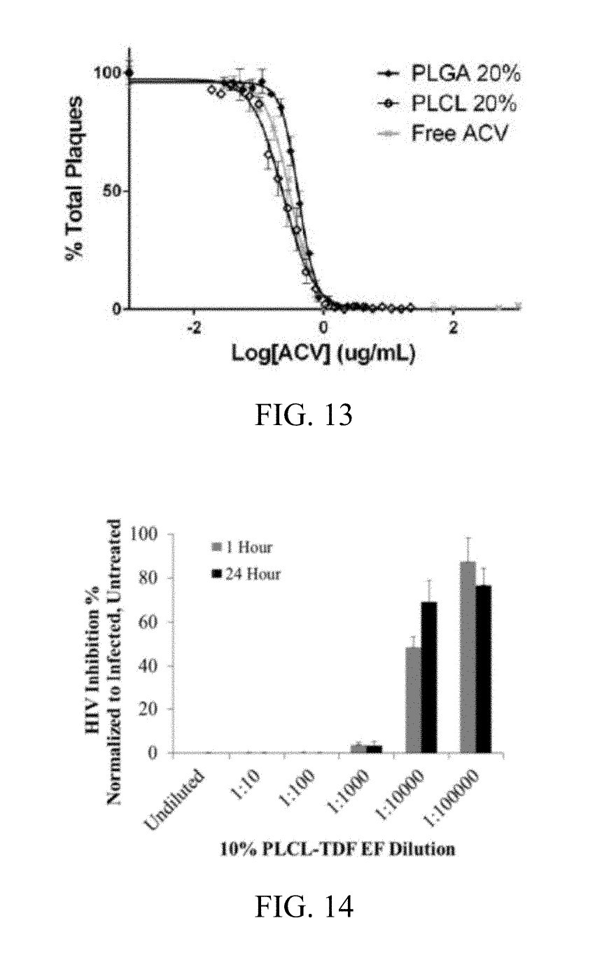

Data demonstrated that EFs that encapsulate and provide sustained-delivery of ACV and TDF can be created under conditions relevant to intravaginal delivery, and that GRFT-modified EFs can be obtained. Another goal was to complete in vitro and tissue safety and efficacy studies with the individual EFs A combinatorial approach, comprised of GRFT-EFs that release both ACV and TDF was expected to be the most effective long-term strategy, and information was thus first sought from in vivo studies to synthesize and test combined EFs. Initially, experiments were undertaken to determine the in vitro efficacy of unmodified EFs that encapsulated antivirals (ACV and TDF) or are surface-modified with GRFT, against HSV-2 infection. With respect to the efficacy against HSV-2 infection, it was found that ACV and TDF EFs completely inhibit HSV-2 infection in vitro. In these experiments, the in vitro efficacy of PLGA and PLCL fibers encapsulating ACV or TDF were evaluated at 3 different concentrations against HSV-2 infection in vitro. FIG. 12 shows the results of a plaque assay experiment using the lowest concentration (1% w/w) of ACV in both PLGA (A, bottom row) and PLCL (B, bottom row) EFs. The top row of each plate contained cells that were incubated with blank PLGA or PLCL EFs. Briefly, Vero E6 cells were incubated at a density of 600,000 cells/well in a 6-well plate, and the next day upon confluence, cells were infected with 1000 pfu/well HSV-2 for 1 hr to enable plaque counting of approximately 200-300 pfu per well in untreated, infected cells. After infection, cells were incubated with IgG to neutralize free HSV-2, and EF eluants were administered to Vero E6 cells. Appropriate controls of: [no treatment/no virus], [no treatment/virus], [blank PLGA or PLCL EFs, virus], and [free ACV, virus] were conducted. FIG. 12 demonstrates that PLGA and PLCL fiber portions that encapsulate 0.1 mg ACV (1% w/w, 3 mg ACV in total 600 mg fiber) inhibit virus completely, relative to blank PLGA and PLCL EFs. Similarly, PLGA and PLCL fibers that contain higher concentrations of 10 and 20 w/w % ACV per fiber, fully inhibited HSV-2 infection.

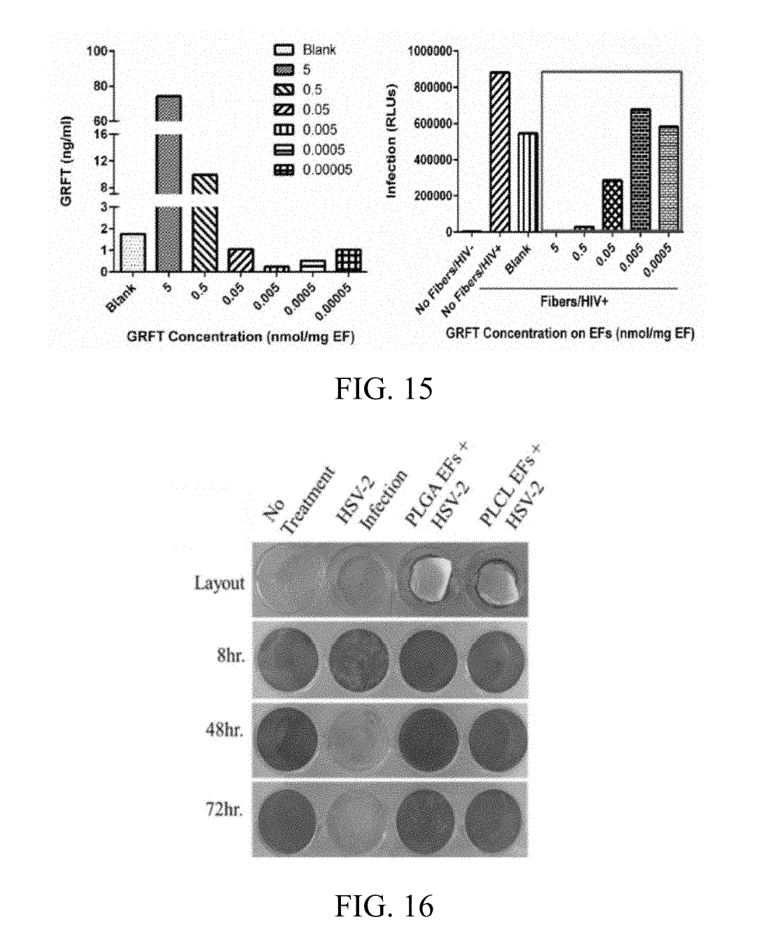

The IC50 (50% inhibitory concentration) of 20% ACV fibers, relative to ACV alone at 3 different release times: 3, 28, and 21-28 days, was also evaluated. The average IC50s using fiber eluant collected after 3 days were 0.393 μg/mL and 0.226 μg/mL for PLGA and PLCL 20% ACV EFs respectively, compared to an average IC50 of 0.287 μg/mL for free ACV. Similarly, fibers were assessed after 28 days to ensure bioactivity after one month in SVF. This resulted in EF IC50 values of 0.426, 0.314, and 0.293 μg/mL for PLGA EFs, PLCL EFs, and free ACV respectively. Last, the effect of eluant released from PLGA and PLCL 20% ACV EFs between 21 and 28 days was evaluated—i.e., during the fourth week of delivery. Similar IC50s of 0.417, 0.314, and 0.293 μg/mL were attained for PLGA, PLCL, and free ACV respectively. All EF formulations achieved comparable bioactivity to free ACV—demonstrating the ability of PLGA and PLCL EFs to: efficiently encapsulate ACV, maintain bioactivity in SVF for up to 28 days, and provide corresponding and complete protection against HSV-2 infection in vitro. Only one of the three IC50 curves is shown for each in FIG. 13 for purposes of brevity. Those results are in agreement with expected in vitro IC50 ranges of ACV (0.03-3.8 Similarly complete HSV-2 inhibition was achieved for all TDF-encapsulated EFs (1, 10, and 20% w/w).

### Example 8—Determination of the In Vitro Efficacy of Unmodified EFs that Encapsulate TDF or GRFT-Modified EFs, Against HIV-1 Infection

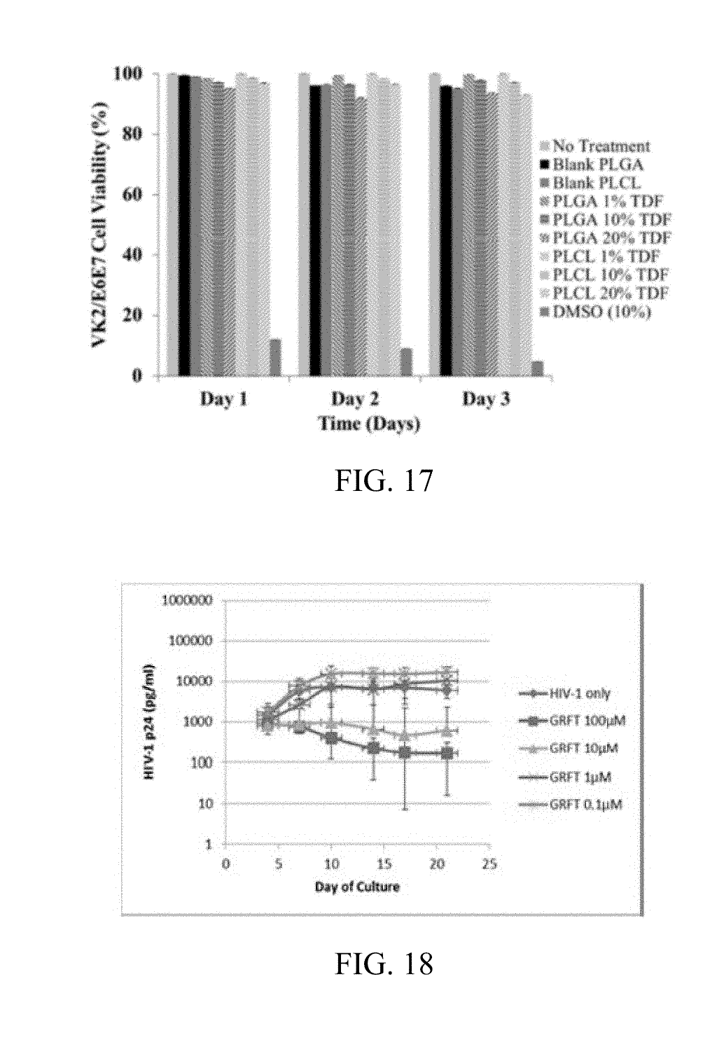

To determine efficacy against HIV-1 infection, HIV-1 infection assays were again conducted in vitro. A reporter gene expression Env-pseudotyped virus infectivity assay was used, as described previously, to measure the HIV-1 antiviral activity of the TDF PLGA and PLCL EFs. Antiviral activity was measured as a reduction in firefly Luciferase (Luc) reporter gene expression after administration of different EF concentrations 1 and 24 hr before a single round of infection in TZM-bl cells (i.e., the engineered HeLa cell clone that stably expresses CD4, CCR5, and CXCR4 receptors and contains HIV-1 Tat-regulated reporter genes for firefly luciferase and B-galactosidase). One set of 8 control wells received only cells plus virus (virus control), and another set of 8 wells received cells only (background control). After 48 hr incubation, culture medium was removed from each well, an equal volume of Bright-Glo™ reagent was added, and luminescence was measured. The EC50 was determined as the TDF-EF concentration that caused a 50% reduction in RLU relative to virus control wells after subtraction of background RLU (FIG. 14). Similar to free TDF, TDF PLGA and PLCL EFs provided complete protection against HIV-1 (FIG. 14).

In connection with the TDF experiments, the neutralizing activity of 6 different concentrations of GRFT-EFs was again similarly assessed against HIV-1 infection. Briefly, TZM-bl cells were seeded in a 96-well plate and grown to approximately 50% confluence overnight prior to infection. GRFT-EFs were added with virus 90 min. prior to HIV-1 infection (300 TCID50/well). After 90 min, media containing unbound virus was removed from the well and added to plated TZM-bl cells. Forty-eight hours later, cells were lysed and luciferase expression was measured and related to the viral standard curve. As shown in FIG. 15, the surface density of GRFT (left) correlated with inhibition (right), with 2 concentrations completely inhibiting HIV infection.

### Example 9—Evaluation of the In Vitro Efficacy of Unmodified Blank EFs to Physically Decrease Virus Penetration

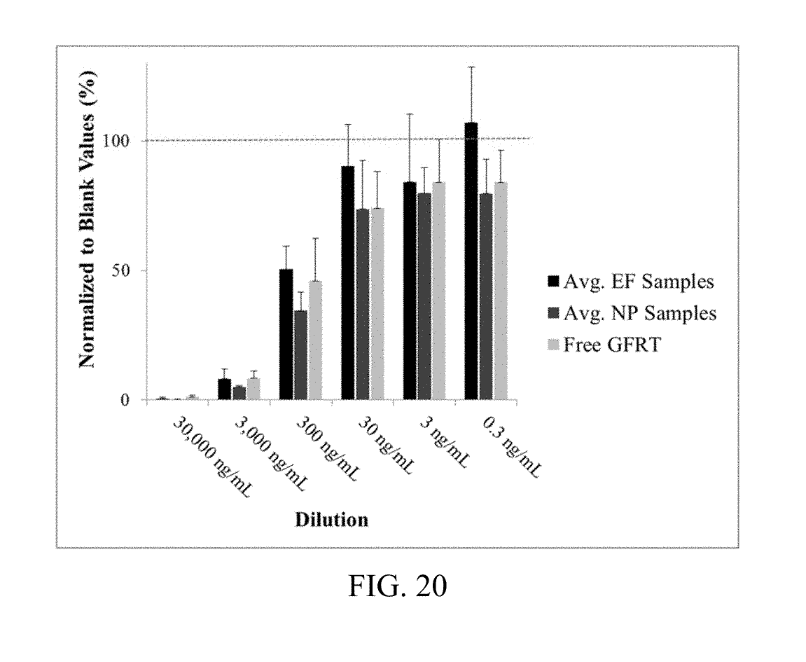

In addition to assessing the antiviral properties of the EFs, experiments were undertaken to investigate if bare EFs can function as a physicochemical barrier to virus penetration. That is, whether unmodified EFs alone can decrease the flux of HSV-2 or HIV-1 passage to underlying infectible cells. To assess HSV-2 penetration through EFs to underlying Vero E6 cells, unmodified blank EFs were inserted in place of a transwell membrane (FIG. 16). Briefly, Vero cells were plated in 12-well plates, and HSV-2 (4674) was added directly upon EFs that were in contact with underlying media and cells. After 2, 4, 6, 12, 24, 48, or 72 hr of virus incubation, the fiber inserts were removed, IgG was added to underlying cells, and 48 hr later viral plaques were counted. Virus infection of cells underlying the unmodified EFs was completely inhibited until day 3.

### Example 10—Determination of the Safety of Individual and Combined EFs in Cell Culture and EpiVaginal™ Tissue Models

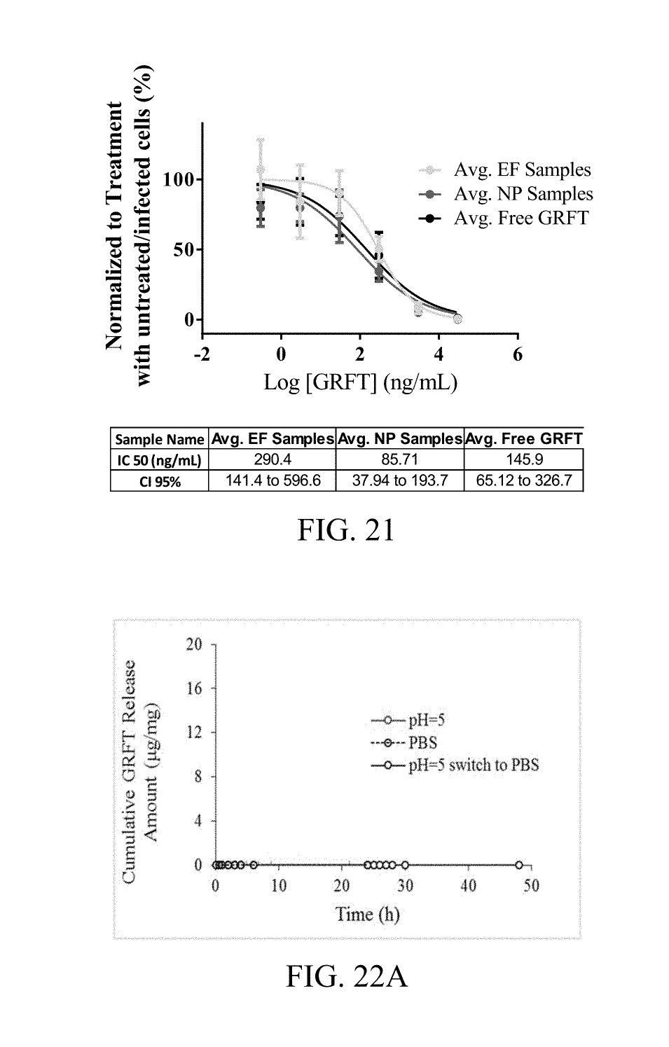

Cells were plated at a density of 300,000 cells per well in a 12-well plate. Cells were incubated in triplicate with 1 mg fiber pieces placed in the transwell inserts (1 mg/mL final concentration) of: 1, 10, and 20% ACV and TDF EFs and 5 nmol/mg GRFT EFs. No treatment (media alone) and 10% DMSO were used as positive and negative controls of cell viability, respectively. After 1, 2, and 3 days incubation, 10 μL of MTT reagent was added to the cells, cells were lysed, and absorbance was read at 570 nm the following day. In all cell lines, all EF-treated cells demonstrated greater than 93% viability, as normalized to untreated cells (FIG. 17).

### Example 11—Determination of the Efficacy of Individual and Combined EFs in EpiVaginal™ Tissue Models

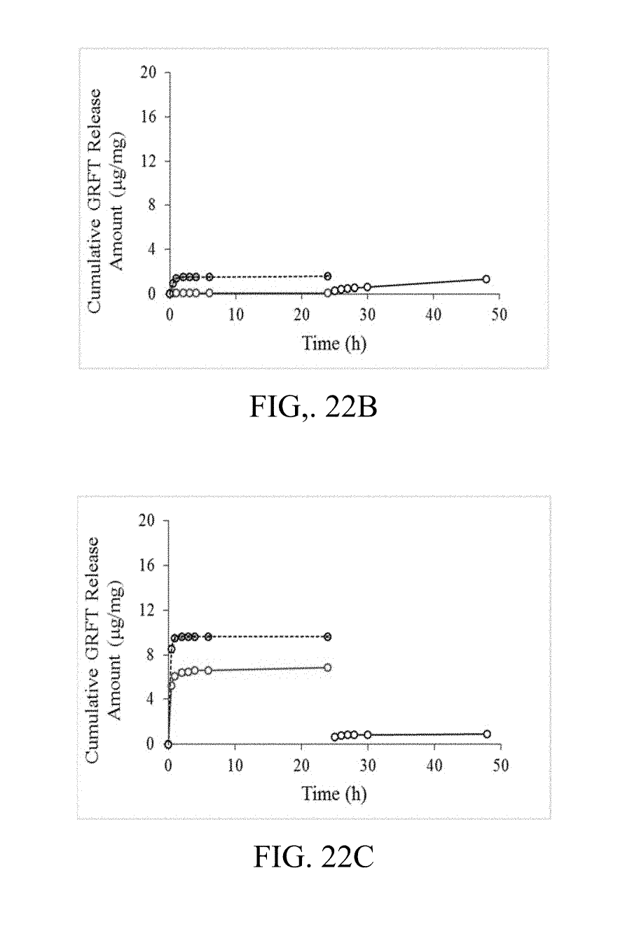

In further experiments, it was found that free GRFT blocks HIV-1 infection in ectocervical explants. In these experiments, the ability of the above-described compositions to prevent HSV-2 and HIV-1 infection was evaluated in the MatTek Epivaginal tissue model and the safety and efficacy of 10 μM GRFT API gel to prevent HIV-1 infection in human cervical explants was demonstrated (FIG. 18).

### Example 12—GRFT-Encapsulated Fibers (EFs) and Nanoparticles (NPs)

PLGA:PBA-co-PAA (Poly (lactic-co-glycolic acid): polybutyl acrylate: polyacrylic acid) electrospun fibers or PLGA nanoparticles were encapsulated with GRFT via electrospinning (fibers) or the double emulsion technique (nanoparticles), respectively. EFs were loaded with 30 μg GRFT per mg polymer, and NPs were loaded with 50 to 200 μg GRFT per mg polymer. In particular, for NP synthesis of encapsulated GRFT, GRFT NPs were synthesized using the double emulsion solvent evaporation technique as previously described. Briefly, 100-200 mg PLGA (50:50 poly(DL-lactide co-glycolide) carboxylate end group or mPEG(5000)-PLGA, inherent viscosity range 0.55-0.75 dL/g, LACTEL®) was dissolved overnight in 1-2 mL dichloromethane (DCM). Two hundred microliters (containing 50-200 μg GRFT) in Tris-EDTA buffer was added dropwise to 100 mg of polymer in solution while vortexing. This solution was sonicated and subsequently added to a 2.5% polyvinyl alcohol (PVA) solution to produce the second emulsion. NPs were hardened during solvent evaporation in 0.3% PVA for 3 hr. The hardened NPs were washed 3 times in deionized water to remove residual solvent, centrifuged at 4° C., lyophilized, and stored at −20° C. until use.

For the synthesis of PLGA and PLCL electrospun fibers, PLGA and PLCL EFs were prepared and electrospun with different solvents and compositions spanning (8-30% wt drug/wt polymer (w/w)) to establish a baseline blank EF (no drug) formulation. For blank polymer EFs, solutions of 10-30% PLGA w/w and 8-12% PLCL w/w were prepared in various solvents (CF:DMF (3:1 and 9:1 vol %), TFE, or HFIP) and allowed to solubilize overnight on a shaker at room temperature. Three milliliters of each polymer solution were aspirated into, and spun from a 3 mL plastic syringe on a custom built device housed in an air-filtered Plexiglas chamber. Flow rates spanning (0.5-3.0 mL/hr) were optimized over a range of voltages (15-27 kV) and the resulting fiber mat was collected on a rotating 4 or 26 mm outer diameter stainless steel mandrel, located 25 cm from the blunt needle tip. Sample flow rate was monitored by an infusion pump (Fisher Scientific, Pittsburgh, Pa.) and the voltage was applied using a high voltage power supply (Spellman CZE 1000R). Final optimized formulations of fibers were spun at 27 kV at 2.0 mL/hr for both PLGA and PLCL formulations. For ACV incorporation, fibers were prepared with 1, 10, and 20% w/w ACV dissolved in polymer solution overnight. After electrospinning, fibers were removed from the mandrel and dried overnight in a desiccator cabinet.

For the synthesis of griffithsin incorporated PLGA/PBA-co-PAA and mPEG-PLGA/PBA-co-PAA polymer blend electrospun fibers, pH-responsive and/or sustained-release polymer blend electrospun fibers: PLGA/PBA-co-PAA and mPEG-PLGA/PBA-co-PAA were fabricated. Briefly, 15˜30% (w/w) mPEG-PLGA were prepared with HFIP, and different amounts of PBA-co-PAA were added to HFIP solvent with various range of blends (w/w): 100:0, 90:10, 85:15, 80:20, and 75:25. Polymers were allowed to dissolve completely overnight on a shaker at room temperature. After desalting GRFT protein stock with Spin-X® UF concentrator (10K MWCO, Corning Incorporated-Life Sciences, Oneonta, N.Y., USA), GRFT was added to the polymer blend solvent solution with 10 to 50 μg/mg theoretical loading. All solutions were extruded from a 3 mL plastic syringe with an 18 G blunt-end needle. One precision syringe pump (Fisher Scientific, Pittsburgh, Pa., USA) was employed to control flow rates. A high voltage power supply (Spellman CZE 1000R, Spellman High Voltage Electronics Corp., Hauppauge, N.Y., USA) was clipped to the needle to provide the charge to the polymer solution. The resulting fiber mat was collected on a 4 mm outer diameter stainless steel grounded mandrel. Fibers were removed from the mandrel and storage in a desiccator cabinet until used for analysis. All electrospinning processes were performed at room temperature (RT; ˜25° C.). The following electrospinning parameters were also optimized: flow rate (0.4-1.0 mL/h), voltage (20-25 kV), distance from needle to mandrel (10-25 cm), and polymer viscosity (polymer concentration 15-30%). For comparison, PLGA/PBA-co-PAA blend fiber was also prepared from the electrospinning method as described above.

For, NP surface-modification, the protocol develop for surface modification of electrospun fibers described above and making use of NHS-EDC chemical crosslinkers was primarily utilized. However, development efforts were also directed toward conjugation of agents utilizing an avidin-palmitate system. In such a system, NP formulations were synthesized by adding (1 mg/mL) avidin-palmitate to the 5% PVA solution during the emulsion process. NPs were collected after the first wash and incubated for 30 min with biotinylated ligands (such as biotinylated GRFT) at a molar ratio of 3:1 ligand:avidin in PBS. After conjugation, the NPs were washed two more times with diH2O by centrifugation and subsequent washing. All NPs were frozen, lyophilized, and stored at −20° C. until use.

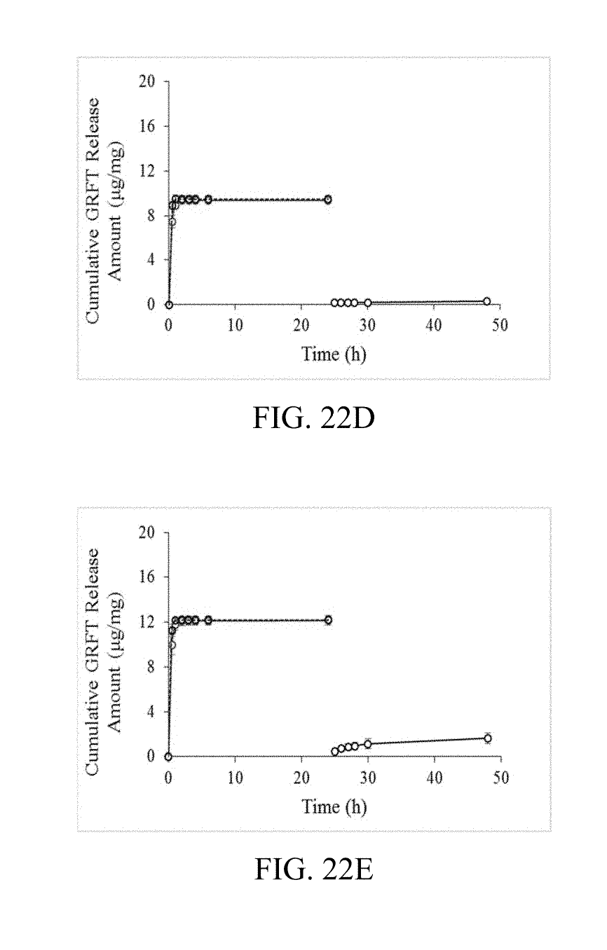

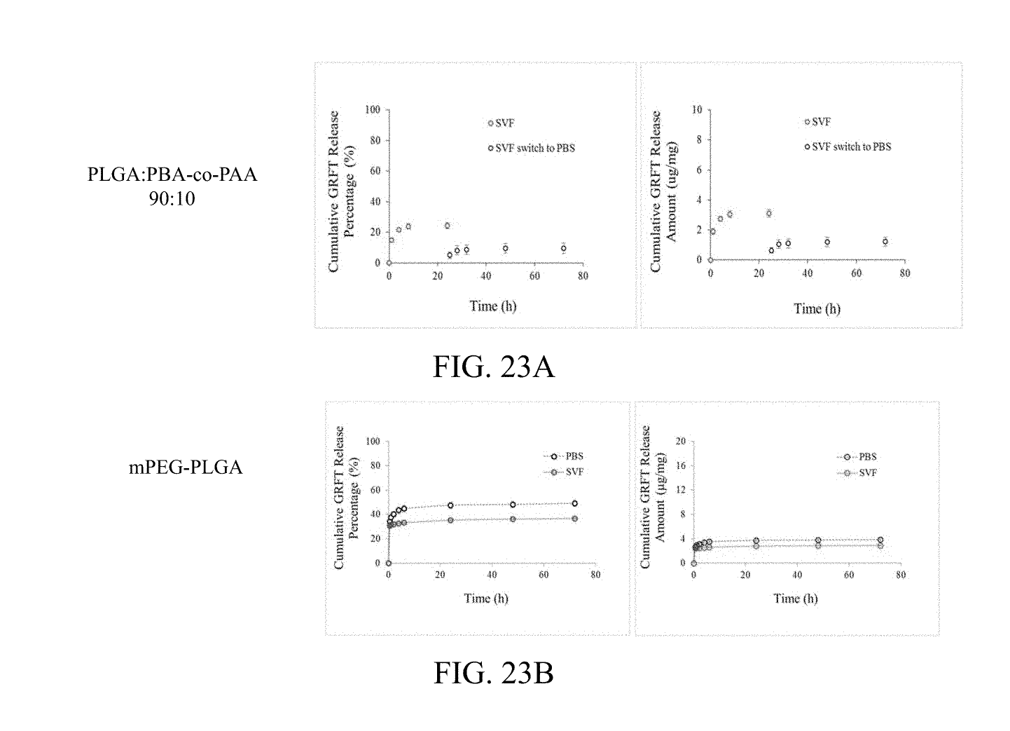

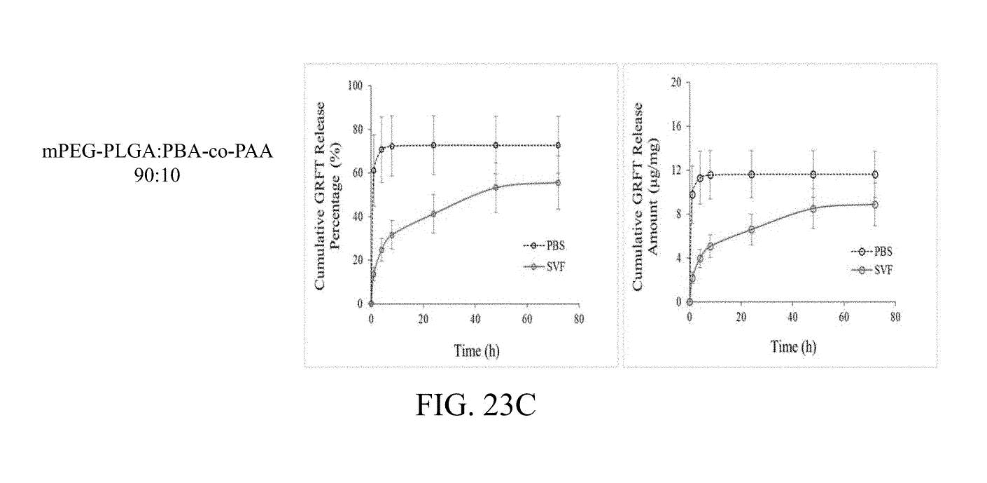

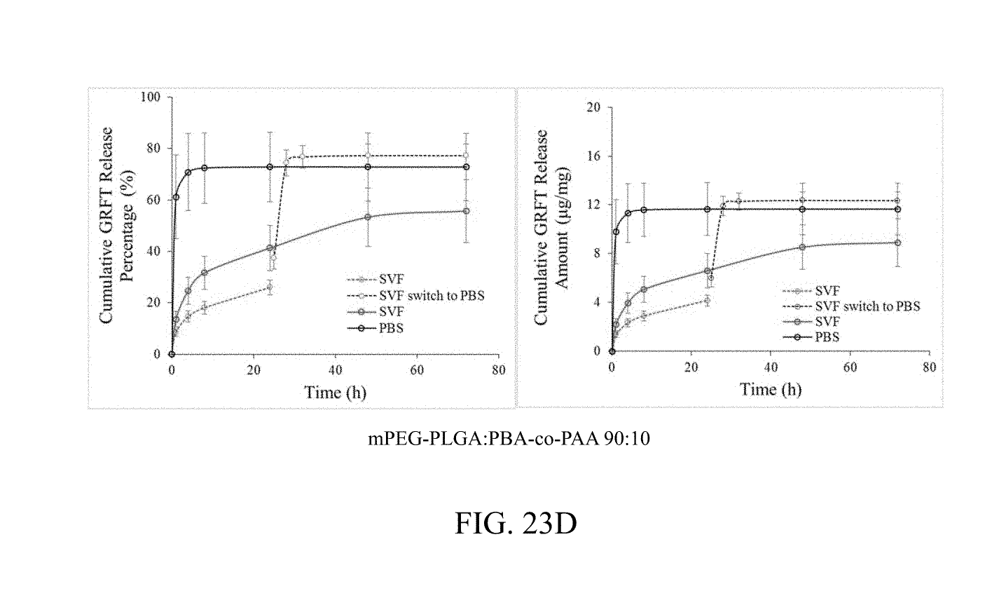

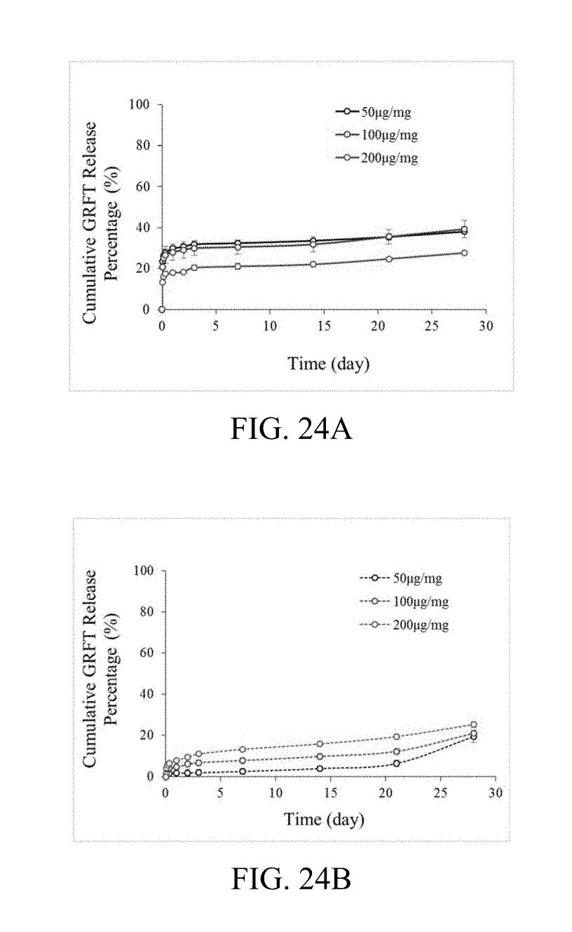

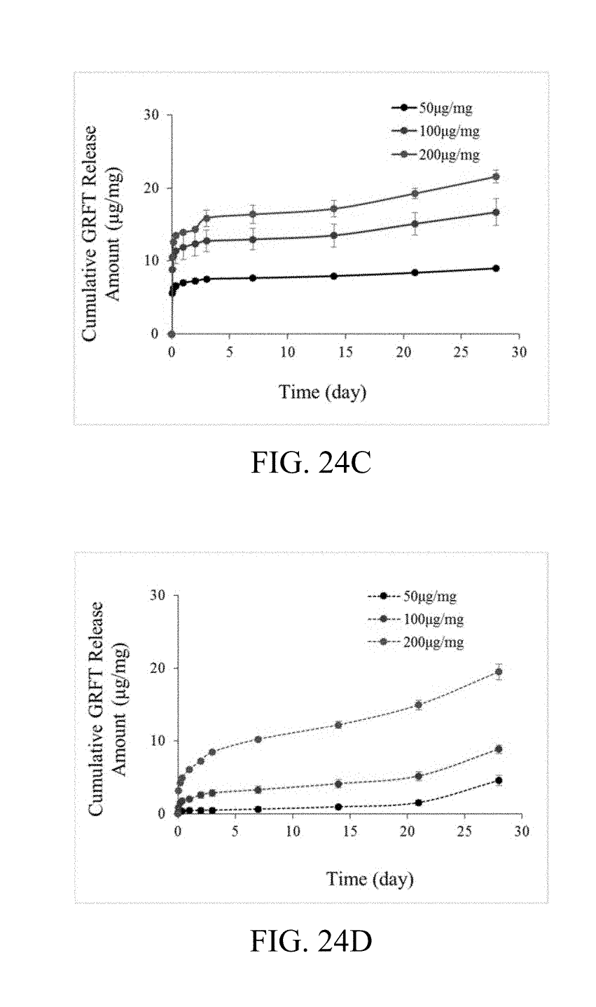

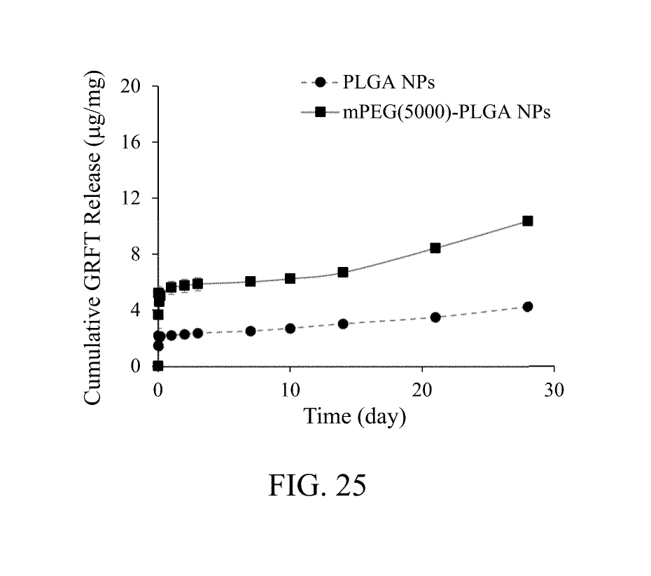

FIGS. 19-20 show dilutions of GRFT extracted from the EFs and NPs (at associated concentrations) and FIG. 21 shows dose-dependent efficacy against HIV-1 infection in vitro. In further experiments evaluating GRFT-encapsulated Fibers (EFs) and nanoparticles (NPs), EFs were also synthesized as PLGA only, various ratios of PLGA:PBA-co-PAA, mPEG(5000)-PLGA, and mPEG(5000)-PLGA:PBA-co-PAA 90:10 as described above, while NPs were synthesized with PLGA, mPEG(5000)-PLGA, and PLGA: 5, 10, and 20% PEG as also described above. Upon synthesis, the release of GRFT from the EFs and NPs was evaluated and it was observed that both compositions afforded tunable release with pH change in a variety of different polymer formulations and in both PBS and SVF (see FIGS. 22A-22E, 23A-23D, 24A-24D, and 25).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Rohan L C, Sassi A B. 2009. Vaginal drug delivery systems for HIV prevention. Aaps J 11:78-87.
2. CDC. 2012. Genital Herpes—CDC Fact Sheet (Detailed).
3. WHO. 2013. World Health Organization, Health Topics HIV/AIDS. Accessed
4. UNAIDS. 2013. Global Report UNAIDS report on the global AIDS epidemic 2013. Accessed
5. Looker K J, Garnett G P, Schmid G P. 2008. An estimate of the global prevalence and incidence of herpes simplex virus type 2 infection. Bull World Health Organ 86:805-812, A.
6. Freeman E E, Weiss H A, Glynn J R, Cross P L, Whitworth J A, Hayes R J. 2006. Herpes simplex virus 2 infection increases HIV acquisition in men and women: systematic review and meta-analysis of longitudinal studies. AIDS 20:73-83.
7. Looker K J, Magaret A S, Turner K M, Vickerman P, Gottlieb S L, Newman L M. 2015. Global estimates of prevalent and incident herpes simplex virus type 2 infections in 2012. PLoS One 10:e114989.
8. Berer M. 2006. Dual protection: more needed than practised or understood. Reprod Health Matters 14:162-170.
9. Brady M. 2003. Preventing sexually transmitted infections and unintended pregnancy, and safeguarding fertility: triple protection needs of young women. Reprod Health Matters 11:134-141.
10. Bull S S, Shlay J C. 2005. Promoting “dual protection” from pregnancy and sexually transmitted disease: a social ecological approach. Health Promot Pract 6:72-80.
11. Cates W, Jr., Steiner M J. 2002. Dual protection against unintended pregnancy and sexually transmitted infections: what is the best contraceptive approach? Sex Transm Dis 29:168-174.
12. Chandran P, Kabir S N. 2010. Dual action microbicides: reappraisal of their roles in contraceptive research. Reprod Biomed Online 20:103-113.
13. Karim Q A, Karim S S A, Frohlich J A, Grobler A C, Baxter C, Mansoor L E, Kharsany A B M, Sibeko S, Mlisana K P, Omar Z, Gengiah T N, Maarschalk S, Arulappan N, Mlotshwa M, Morris L, Taylor D, Grp C T. 2010. Effectiveness and Safety of Tenofovir Gel, an Antiretroviral Microbicide, for the Prevention of HIV Infection in Women. Science 329:1168-1174.
14. CONRAD. 2014. CONRAD: Clinical Trials. Antiretrovirals as Microbides: Tenofovir (TFV).
15. IPM. 2014. International Partnership for Microbicides Webpage.
16. MTN. 2011. MTN statement on decision to discontinue use of oral tenofovir tablets in VOICE, a major HIV prevention study in women.
17. MTN. 2016. Dapivirine vaginal ring helped protect women against HIV in ASPIRE Phase III trial.
18. Network M T. 2014. MTN: Microbicide Trials Network—Studies.
19. Steinbach J M, Seo Y E, Saltzman W M. 2015. Cell penetrating peptide-modified poly(lactic-co-glycolic acid) nanoparticles with enhanced cell internalization. Acta Biomater doi:10.1016/j.actbio.2015.11.029.
20. Lai S K, Wang Y Y, Hanes J. 2009. Mucus-penetrating nanoparticles for drug and gene delivery to mucosal tissues. Adv Drug Deliv Rev 61:158-171.
21. Lai S K, Wang Y Y, Hida K, Cone R, Hanes J. 2010. Nanoparticles reveal that human cervicovaginal mucus is riddled with pores larger than viruses. Proc Natl Acad Sci USA 107:598-603.
22. Lai S K, Wang Y Y, Wirtz D, Hanes J. 2009. Micro- and macrorheology of mucus. Adv Drug Deliv Rev 61:86-100.
23. Oh M J, Akhtar J, Desai P, Shukla D. 2010. A role for heparan sulfate in viral surfing. Biochem Biophys Res Commun 391:176-181.
24. Cu Y, Saltzman W M. 2009. Mathematical modeling of molecular diffusion through mucus. Adv Drug Deliv Rev 61:101-114.
25. Cu Y, Saltzman W M. 2009. Controlled surface modification with poly(ethylene)glycol enhances diffusion of PLGA nanoparticles in human cervical mucus. Mol Pharm 6:173-181.
26. Cu Y, Saltzman W M. 2009. Drug delivery: Stealth particles give mucus the slip. Nat Mater 8:11-13.
27. Ensign L M, Tang B C, Wang Y Y, Tse T A, Hoen T, Cone R, Hanes J. 2012. Mucus-penetrating nanoparticles for vaginal drug delivery protect against herpes simplex virus. Sci Transl Med 4:138ra179.
28. Tang B C, Dawson M, Lai S K, Wang Y Y, Suk J S, Yang M, Zeitlin P, Boyle M P, Fu J, Hanes J. 2009. Biodegradable polymer nanoparticles that rapidly penetrate the human mucus barrier. Proc Natl Acad Sci USA 106:19268-19273.
29. Liang D, Hsiao B S, Chu B. 2007. Functional electrospun nanofibrous scaffolds for biomedical applications. Adv Drug Deliv Rev 59:1392-1412.
30. Chou S F, Carson D, Woodrow K A. 2015. Current strategies for sustaining drug release from electrospun nanofibers. J Control Release 220:584-591.
31. Ayres C E, Jha B S, Sell S A, Bowlin G L, Simpson D G. 2010. Nanotechnology in the design of soft tissue scaffolds: innovations in structure and function. Wiley Interdiscip Rev Nanomed Nanobiotechnol 2:20-34.
32. Wan-Ju L, Shanti R M, Tuan R S. 2006. Electrospinning Technology for Nanofibrous Scaffolds in Tissue Engineering, Nanotechnologies for the Life Sciences, vol 9. Wiley, Tissue, Cell and Organ Engineering.
33. Huang C, Soenen S J, van Gulck E, Rejman J, Vanham G, Lucas B, Geers B, Braeckmans K, Shahin V, Spanoghe P, Demeester J, De Smedt S. 2014. Electrospun polystyrene fibers for HIV entrapment. Polymers Advanced Technologies 25:827-834.
34. Zamani M, Prabhakaran M P, Ramakrishna S. 2013. Advances in drug delivery via electrospun and electrosprayed nanomaterials. Int J Nanomedicine 8:2997-3017.
35. Ball C, Krogstad E, Chaowanachan T, Woodrow K A. 2012. Drug-eluting fibers for HIV-1 inhibition and contraception. PLoS One 7:e49792.
36. Blakney A K, Ball C, Krogstad E A, Woodrow K A. 2013. Electrospun fibers for vaginal anti-HIV drug delivery. Antiviral Res 100 Suppl:S9-16.
37. Huang C, Soenen S J, van Gulck E, Vanham G, Rejman J, Van Calenbergh S, Vervaet C, Coenye T, Verstraelen H, Temmerman M, Demeester J, De Smedt S C. 2012. Electrospun cellulose acetate phthalate fibers for semen induced anti-HIV vaginal drug delivery. Biomaterials 33:962-969.
38. Aniagyei S E, Sims L B, Malik D A, Kim W, Steinbach J M. submitted. Electrospun Polymer Nanofibers to Inhibit HSV-2 Infection.
39. Steinbach J M. 2015. Protein and oligonucleotide delivery systems for vaginal microbicides against viral STIs. Cell Mol Life Sci 72:469-503.
40. Ball C, Chou S F, Jiang Y, Woodrow K A. 2016. Coaxially electrospun fiber-based microbicides facilitate broadly tunable release of maraviroc. Mater Sci Eng C Mater Biol Appl 63:117-124.
41. Carson D, Jiang Y, Woodrow K A. 2016. Tunable Release of Multiclass Anti-HIV Drugs that are Water-Soluble and Loaded at High Drug Content in Polyester Blended Electrospun Fibers. Pharm Res 33:125-136.
42. Danhier F, Ansorena E, Silva J M, Coco R, Le Breton A, Preat V. 2012. PLGA-based nanoparticles: an overview of biomedical applications. J Control Release 161:505-522.
43. Woodruff M A, Hutmacher D W. 2010. The return of a forgotten polymer: Polycaprolactone in the 21st century. Progress in Polymer Science 35:1217-1256.
44. Makadia H K, Siegel S J. 2011. Poly Lactic-co-Glycolic Acid (PLGA) as Biodegradable Controlled Drug Delivery Carrier. Polymers (Basel) 3:1377-1397.
45. Xue J, Hoorelbeke B, Kagiampakis I, Demeler B, Balzarini J, Liwang P J. 2013. The griffithsin dimer is required for high-potency inhibition of HIV-1: evidence for manipulation of the structure of gp120 as part of the griffithsin dimer mechanism. Antimicrob Agents Chemother 57:3976-3989.
46. Meuleman P, Albecka A, Belouzard S, Vercauteren K, Verhoye L, Wychowski C, Leroux-Roels G, Palmer K E, Dubuisson J. 2011. Griffithsin has antiviral activity against hepatitis C virus. Antimicrob Agents Chemother 55:5159-5167.
47. Nixon B, Stefanidou M, Mesquita P M, Fakioglu E, Segarra T, Rohan L, Halford W, Palmer K E, Herold B C. 2013. Griffithsin protects mice from genital herpes by preventing cell-to-cell spread. J Virol 87:6257-6269.
48. O'Keefe B R, Giomarelli B, Barnard D L, Shenoy S R, Chan P K, McMahon J B, Palmer K E, Barnett B W, Meyerholz D K, Wohlford-Lenane C L, McCray P B, Jr. 2010. Broad-spectrum in vitro activity and in vivo efficacy of the antiviral protein griffithsin against emerging viruses of the family Coronaviridae. J Virol 84:2511-2521.
49. Ishag H Z, Li C, Huang L, Sun M X, Wang F, Ni B, Malik T, Chen P Y, Mao X. 2013. Griffithsin inhibits Japanese encephalitis virus infection in vitro and in vivo. Arch Virol 158:349-358.
50. Ferir G, Huskens D, Palmer K E, Boudreaux D M, Swanson M D, Markovitz D M, Balzarini J, Schols D. 2012. Combinations of griffithsin with other carbohydrate-binding agents demonstrate superior activity against HIV Type 1, HIV Type 2, and selected carbohydrate-binding agent-resistant HIV Type 1 strains. AIDS Res Hum Retroviruses 28:1513-1523.
51. O'Keefe B R, Vojdani F, Buffa V, Shattock R J, Montefiori D C, Bakke J, Mirsalis J, d'Andrea A L, Hume S D, Bratcher B, Saucedo C J, McMahon J B, Pogue G P, Palmer K E. 2009. Scaleable manufacture of HIV-1 entry inhibitor griffithsin and validation of its safety and efficacy as a topical microbicide component. Proc Natl Acad Sci USA 106:6099-6104.
52. Mori T, O'Keefe B R, Sowder R C, 2nd, Bringans S, Gardella R, Berg S, Cochran P, Turpin J A, Buckheit R W, Jr., McMahon J B, Boyd M R. 2005. Isolation and characterization of griffithsin, a novel HIV-inactivating protein, from the red alga Griffithsia sp. J Biol Chem 280:9345-9353.
53. Emau P, Tian B, O'Keefe B R, Mori T, McMahon J B, Palmer K E, Jiang Y, Bekele G, Tsai C C. 2007. Griffithsin, a potent HIV entry inhibitor, is an excellent candidate for anti-HIV microbicide. J Med Primatol 36:244-253.
54. Ferir G, Palmer K E, Schols D. 2011. Synergistic activity profile of griffithsin in combination with tenofovir, maraviroc and enfuvirtide against HIV-1 clade C. Virology 417:253-258.
55. Hamorsky K T, Grooms-Williams T W, Husk A S, Bennett L J, Palmer K E, Matoba N. 2013. Efficient single tobamoviral vector-based bioproduction of broadly neutralizing anti-HIV-1 monoclonal antibody VRC01 in Nicotiana benthamiana plants and utility of VRC01 in combination microbicides. Antimicrob Agents Chemother 57:2076-2086.
56. B R, Wlodawer A. 2010. Monomerization of viral entry inhibitor griffithsin elucidates the relationship between multivalent binding to carbohydrates and anti-HIV activity. Structure 18:1104-1115.
57. Kouokam J C, Huskens D, Schols D, Johannemann A, Riedell S K, Walter W, Walker J M, Matoba N, O'Keefe B R, Palmer K E. 2011. Investigation of griffithsin's interactions with human cells confirms its outstanding safety and efficacy profile as a microbicide candidate. PLoS One 6:e22635.
58. Barton C, Kouokam J C, Lasnik A B, Foreman O, Cambon A, Brock G, Montefiori D C, Vojdani F, McCormick A A, O'Keefe B R, Palmer K E. 2014. Activity of and effect of subcutaneous treatment with the broad-spectrum antiviral lectin griffithsin in two laboratory rodent models. Antimicrob Agents Chemother 58:120-127.
59. Ziolkowska N E, O'Keefe B R, Mori T, Zhu C, Giomarelli B, Vojdani F, Palmer K E, McMahon J B, Wlodawer A. 2006. Domain-swapped structure of the potent antiviral protein griffithsin and its mode of carbohydrate binding. Structure 14:1127-1135.
60. Ziolkowska N E, Shenoy S R, O'Keefe B R, McMahon J B, Palmer K E, Dwek R A, Wormald M R, Wlodawer A. 2007. Crystallographic, thermodynamic, and molecular modeling studies of the mode of binding of oligosaccharides to the potent antiviral protein griffithsin. Proteins 67:661-670.
61. Fuqua J L, Wanga V, Palmer K E. 2015. Improving the large scale purification of the HIV microbicide, griffithsin. BMC Biotechnol 15:12.
62. Owen D H, Katz D F. 1999. A vaginal fluid stimulant. Contraception 59:91-95.
63. Montefiori D C. 2009. Measuring HIV neutralization in a luciferase reporter gene assay. Methods Mol Biol 485:395-405.
64. Zhang P F, Chen X, Fu D W, Margolick J B, Quinnan G V, Jr. 1999. Primary virus envelope cross-reactivity of the broadening neutralizing antibody response during early chronic human immunodeficiency virus type 1 infection. J Virol 73:5225-5230.
65. Boyd M R, Gustafson K R, McMahon J B, Shoemaker R H, O'Keefe B R, Mori T, Gulakowski R J, Wu L, Rivera M I, Laurencot C M, Currens M J, Cardellina J H, 2nd, Buckheit R W, Jr., Nara P L, Pannell L K, Sowder R C, 2nd, Henderson L E. 1997. Discovery of cyanovirin-N, a novel human immunodeficiency virus-inactivating protein that binds viral surface envelope glycoprotein gp120: potential applications to microbicide development. Antimicrob Agents Chemother 41:1521-1530.
66. Wei X, Decker J M, Liu H, Zhang Z, Arani R B, Kilby J M, Saag M S, Wu X, Shaw G M, Kappes J C. 2002. Emergence of resistant human immunodeficiency virus type 1 in patients receiving fusion inhibitor (T-20) monotherapy. Antimicrob Agents Chemother 46:1896-1905.
67. Platt E J, Wehrly K, Kuhmann S E, Chesebro B, Kabat D. 1998. Effects of CCR5 and CD4 cell surface concentrations on infections by macrophagetropic isolates of human immunodeficiency virus type 1. J Virol 72:2855-2864.
68. Montefiori D C. 2011. Protocol for the Preparation and Titration of HIV-1 Env-pseudotyped Viruses. Accessed
69. ClinicalTrials.gov. 2015. Phase 3 Safety and Effectiveness Trial of Dapivirine Vaginal Ring for Prevention of HIV-1 in Women (ASPIRE). ClinicalTrials.gov.
70. NIH. 2016. The ASPIRE HIV Prevention Study—Questions and Answers, NIH: National Institute of Allergy and Infectious Diseases.
71. NIH. 2016. Vaginal Ring Provides Partial Protection from HIV in Large Multinational Trial. NIH: National Institute of Allergy and Infectious Disease.
72. Abdool Karim Q, Abdool Karim S S, Frohlich J A, Grobler A C, Baxter C, Mansoor L E, Kharsany A B, Sibeko S, Mlisana K P, Omar Z, Gengiah T N, Maarschalk S, Arulappan N, Mlotshwa M, Morris L, Taylor D. 2010. Effectiveness and safety of tenofovir gel, an antiretroviral microbicide, for the prevention of HIV infection in women. Science 329:1168-1174.
73. Gengiah T N, Abdool Karim Q. 2012. Implementing microbicides in low-income countries. Best Pract Res Clin Obstet Gynaecol 26:495-501.
74. Gengiah T N, Baxter C, Mansoor L E, Kharsany A B, Abdool Karim S S. 2012. A drug evaluation of 1% tenofovir gel and tenofovir disoproxil fumarate tablets for the prevention of HIV infection. Expert Opin Investig Drugs 21:695-715.
75. van der Straten A, Stadler J, Montgomery E, Hartmann M, Magazi B, Mathebula F, Schwartz K, Laborde N, Soto-Torres L. 2014. Women's experiences with oral and vaginal pre-exposure prophylaxis: the VOICE-C qualitative study in Johannesburg, South Africa. PLoS One 9:e89118.
76. MTN. 2015. MTN-003—VOICE.
77. Ball C, Woodrow K A. 2014. Electrospun solid dispersions of Maraviroc for rapid intravaginal preexposure prophylaxis of HIV. Antimicrob Agents Chemother 58:4855-4865.
78. Blakney A K, Krogstad E A, Jiang Y H, Woodrow K A. 2014. Delivery of multipurpose prevention drug combinations from electrospun nanofibers using composite microarchitectures. Int J Nanomedicine 9:2967-2978.
79. Nixon, B. et al. Griffithsin protects mice from genital herpes by preventing cell-to-cell spread. J Virol 87, 6257-69 (2013).
80. Emau, P. et al. Griffithsin, a potent HIV entry inhibitor, is an excellent candidate for anti-HIV microbicide. J Med Primatol 36, 244-53 (2007).
81. Barton, C. et al. Activity of and effect of subcutaneous treatment with the broad-spectrum antiviral lectin griffithsin in two laboratory rodent models. Antimicrob Agents Chemother 58, 120-7 (2014).
82. Mori, T. et al. Isolation and characterization of griffithsin, a novel HIV-inactivating protein, from the red alga Griffithsia sp. J Biol Chem 280, 9345-53 (2005).
83. Moulaei, T. et al. Monomerization of viral entry inhibitor griffithsin elucidates the relationship between multivalent binding to carbohydrates and anti-HIV activity. Structure 18, 1104-15 (2010).
84. Ziolkowska, N. E. et al. Domain-swapped structure of the potent antiviral protein griffithsin and its mode of carbohydrate binding. Structure 14, 1127-35 (2006).
85. Ziolkowska, N. E. et al. Crystallographic, thermodynamic, and molecular modeling studies of the mode of binding of oligosaccharides to the potent antiviral protein griffithsin. Proteins 67, 661-70 (2007).
86. Kouokam, J. C. et al. Investigation of griffithsin's interactions with human cells confirms its outstanding safety and efficacy profile as a microbicide candidate. PLoS One 6, e22635 (2011).
87. Zeitlin, L., Pauly, M. & Whaley, K. J. Second-generation HIV microbicides: continued development of griffithsin. Proc Natl Acad Sci USA 106, 6029-30 (2009).
88. Xue, J. et al. The griffithsin dimer is required for high-potency inhibition of HIV-1: evidence for manipulation of the structure of gp120 as part of the griffithsin dimer mechanism. Antimicrob Agents Chemother 57, 3976-89 (2013).
89. Meuleman, P. et al. Griffithsin has antiviral activity against hepatitis C virus. Antimicrob Agents Chemother 55, 5159-67 (2011).
90. O'Keefe, B. R. et al. Broad-spectrum in vitro activity and in vivo efficacy of the antiviral protein griffithsin against emerging viruses of the family Coronaviridae. J Virol 84, 2511-21 (2010).
91. Ishag, H. Z. et al. Griffithsin inhibits Japanese encephalitis virus infection in vitro and in vivo. Arch Virol 158, 349-58 (2013).
92. Ferir, G. et al. Combinations of griffithsin with other carbohydrate-binding agents demonstrate superior activity against HIV Type 1, HIV Type 2, and selected carbohydrate-binding agent-resistant HIV Type 1 strains. AIDS Res Hum Retroviruses 28, 1513-23 (2012).
93. Ferir, G., Palmer, K. E. & Schols, D. Synergistic activity profile of griffithsin in combination with tenofovir, maraviroc and enfuvirtide against HIV-1 clade C. Virology 417, 253-8 (2011).
94. Ferir, G., K., P. & Schols, D. Griffithsin, Alone and Combined with All Classes of Antiretroviral Drugs, Potently Inhibits HIV Cell-Cell Transmission and Destruction of CD4+ Tcells. Antivirals and Antiretrovirals 4, 103-112 (2012).
95. O'Keefe, B. R. et al. Scaleable manufacture of HIV-1 entry inhibitor griffithsin and validation of its safety and efficacy as a topical microbicide component. Proc Natl Acad Sci USA 106, 6099-104 (2009).
96. Hamorsky, K. T. et al. Efficient single tobamoviral vector-based bioproduction of broadly neutralizing anti-HIV-1 monoclonal antibody VRC01 in Nicotiana benthamiana plants and utility of VRC01 in combination microbicides. Antimicrob Agents Chemother 57, 2076-86 (2013).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A composition, comprising:
an electrospun fiber having a surface; and
a biological adhesive moiety conjugated to the surface of the electrospun fiber.

2. The composition of claim 1, wherein the electrospun fiber is comprised of a biodegradable polymer.

3. The composition of claim 2, wherein the biodegradable polymer is selected from the group consisting of poly(lactic-co-glycolic acid) (PLGA), poly(L-lactide-ε-caprolactone) (PLCL), polybutyl acrylate, polyacrylic acid, and combinations thereof.

4. The composition of claim 3, wherein the electrospun fiber is comprised of PLGA.

5. The composition of claim 1, wherein the biological adhesive moiety is a lectin.

6. The composition of claim 4, wherein the lectin is griffithsin (GRFT).

7. The composition of claim 6, wherein the composition comprises griffithsin at a concentration of about 0.00005 nmol to about 5 nmol per mg of the electrospun fiber.

8. The composition of claim 1, wherein the biological adhesive moiety is conjugated to the surface of the electrospun fiber with a chemical crosslinker.

9. The composition of claim 8, wherein the chemical crosslinker comprises carbodiimide.

10. The composition of claim 1, further comprising one or more antiviral agents.

11. The composition of claim 10, wherein the antiviral agent is selected from acyclovir (ACV), tenofovir (TFV), tenofovir disoproxil fumarate (TFD), and combinations thereof.

12. The composition of claim 10, wherein the antiviral agent is encapsulated by the electrospun fiber.

13. The composition of claim 10, wherein the electrospun fiber comprises a first electrospun fiber and a second electrospun fiber, wherein the biological adhesive moiety is conjugated to the first electrospun fiber, and wherein the one or more antiviral agents are encapsulated by the second electrospun fiber.

14. The composition of claim 10, further comprising one or more polymer nanoparticles, each of the one or more polymer nanoparticles conjugated to an antiviral agent, to a biological adhesive moiety, or to both.

15. The composition of claim 14, wherein the one or more polymer nanoparticles are conjugated to a biological adhesive moiety, and wherein the biological adhesive moiety comprises griffithsin.

16. A method for treating a viral infection, comprising administering to a subject an effective amount of a composition including an electrospun fiber having a surface and a biological adhesive moiety conjugated to the surface of the electrospun fiber.

17. The method of claim 16, wherein administering the composition comprises intravaginally or intranasally administering the composition.

18. The method of claim 16, wherein the biological adhesive moiety is a lectin.

19. The method of claim 18, wherein the lectin is griffithsin (GRFT).

20. The method of claim 16, wherein the composition further comprises one or more antiviral agents.

21. The method of claim 20, wherein the antiviral agent is selected from acyclovir (ACV), tenofovir (TFV), tenofovir disoproxil fumarate (TFD), and combinations thereof.

22. The method of claim 20, wherein the antiviral agent is encapsulated by the electrospun fiber.

23. The method of claim 20, wherein the electrospun fiber comprises a first electrospun fiber and a second electrospun fiber, wherein the biological adhesive moiety is conjugated to the first electrospun fiber, and wherein the one or more antiviral agents are encapsulated by the second electrospun fiber.

24. The method of claim 20, further comprising one or more polymer nanoparticles, each of the one or more polymer nanoparticles conjugated to an antiviral agent, to a biological adhesive moiety, or to both.

25. The method of claim 16, wherein the viral infection is selected from a herpes simplex virus 2 infection, a human immunodeficiency virus infection, a hepatitis C virus infection, a middle east respiratory virus syndrome coronavirus infection, a severe acute respiratory syndrome coronavirus infection, an ebola virus infection, a human papilloma virus infection, an influenza virus infection, an enterovirus infection, a measles virus infection, a simian immunodeficiency virus infection, a human T-lymphotrophic virus infection, and a Japanese encephalitis virus infection.

26. A composition, comprising:
a polymeric nanoparticle having a surface; and
a biological adhesive moiety conjugated to the surface of the polymeric nanoparticle.
